(19) United States
(12) Patent Application Publication
(10) Pub. No.: US 2021/0059977 A1
(43) Pub. Date: Mar. 4, 2021

(54) COMBINATION THERAPIES FOR THE TREATMENT OF AMYOTROPHIC LATERAL SCLEROSIS AND RELATED DISORDERS

(71) Applicant: The General Hospital Corporation, Boston, MA (US)

(72) Inventor: David R. Elmaleh, Newton, MA (US)

(21) Appl. No.: 17/046,186

(22) PCT Filed: Apr. 9, 2019
(86) PCT No.: PCT/US19/26521

§ 371 Date: Oct. 8, 2020

Related U.S. Application Data
(60) Provisional application No. 62/654,772, filed on Apr. 9, 2018.

(51) Int. Cl. A61K 31/352; A61P 25/28

(52) U.S. Cl. CPC A61K 31/352; A61K 45/06; A61P 25/28

(57) ABSTRACT

Described herein are methods of treating neuron inflammation conditions, for example, amyotropic lateral sclerosis and prion disease, comprising administering a therapeutically effective amount of a combination of cromolyn or a cromolyn derivative compound and an anti-inflammatory agent.

### CROSS-REFERENCE TO RELATED APPLICATIONS

This application claims priority to U.S. Provisional Patent Application No. 62/654,772, filed Apr. 9, 2018; the entire contents of which are incorporated herein by reference.

### BACKGROUND

Amyotrophic lateral sclerosis (ALS), also known as Lou Gehrig's disease, is a specific disease which causes the death of neurons controlling voluntary muscles. ALS is characterized by stiff muscles, muscle twitching, and gradually worsening weakness due to muscles decreasing in size. This results in difficulty speaking, swallowing, breathing, and eventual death.

It is estimated that ALS affects as many as 30,000 people in the United States, with 5,000 new cases diagnosed each year. Most people who develop ALS are between the ages of 40 and 70, although the disease can occur at a younger age. Worldwide ALS prevalence is not correlated with racial, ethnic, or socioeconomic groups. It is also estimated that ALS is responsible for 5 of 100,000 deaths in people aged 20 and older. The rate of incidence is about 1-2 per 100,000. Most ALS cases are sporadic, only about 5-10% of the cases are familial ALS. ALS is most common among persons over age 60. Males develop the disease more than females at roughly a 2:1 rate. Fifty percent of subjects die within 3 years.

The incidence of ALS is five times higher than Huntington's disease and about equal to multiple sclerosis.

The main neuro-pathologies associated with ALS are: loss of motor neurons in the spinal cord and diffuse sclerosis of the spinal cord. The proposed pathogenic mechanisms include motor neuron damage as a result of oxidative stress that is not necessarily linked to gene mutation (e.g., SOD1), glutamate mediated excitotoxicity, production of free radicals, increased intracellular calcium, decreased EAAT2 function, abnormal protein aggregation (including Bunina bodies, and neurofilament rich hyaline inclusions where mutants can misfold and co-precipitate with other molecules), and increases in caspase-1 and -9 activation as signs of apoptosis. In summary, ALS is caused by a combination of genetic susceptibility and environmental triggers.

In many studies in ALS patients, immune response abnormalities, including increased levels of antibodies, chemokines, T-cells, and gated calcium channels, as well as other markers of inflammation were observed. ALS patients showed higher levels of circulating chemokines and cytokines, such as MCP-1, IL-17 ALS, and IL-6.

In subjects with a healthy central nervous system (CNS), microglia provide immune surveillance. In response to injury, microglia are activated and produce pro-inflammatory cytokines, reactive nitrating intermediates, reactive oxygenating intermediates, and glutamate. These cause neurons in the inflammatory area to degenerate by an apoptotic mechanism. The protective aspects of inflammation include clearance of debris by microglia, which is important in repair and interaction with T cells.

Early activation of monocytes and microglia has potential to decelerate neurodegenerative progression by modulating immune responses to increase the intrinsic phagocytic capacity of monocytes and microglia without triggering secretion of pro-inflammatory cytokines that could worsen neurodegeneration.

It is known that the changes in the properties of microglia depend on their response to different stimuli in their microenvironment (e.g. cytokines), resulting in a range of phenotypes. Based on the changes in expression of cytokines, receptors, and other markers, monocyte and macrophage states have been defined as: classical activation (M1), alternative activation (M2a), type II alternative activation (M2b), and acquired deactivation (M2c).

Microglia are activated in response to the presence of interferon-γ (IFNγ), tumor necrosis factor alpha (TNFα) from T cells, or antigen-presenting cells. M1 activated microglia can produce reactive oxygen species and result in increased production of pro-inflammatory cytokines such as TNFα and interleukin (IL)-1β.

Macrophage M2 activation is associated with mediators that are known to contribute to the anti-inflammatory actions and reorganization of extracellular matrix. Microglia with M2a phenotypes have increased phagocytosis and produce growth factors such as insulin-like growth factor-1 and anti-inflammatory cytokines such as IL-10. Stimulation of macrophages by IL-4 and/or IL-13 results in an M2a state, sometimes called a wound-healing macrophage and it is generally characterized by low production of pro-inflammatory cytokines (IL-1, TNF and IL-6). The M2a responses are primarily observed in allergic responses, extracellular matrix deposition, and remodeling.

M2b macrophages are unique in that they express high levels of pro-inflammatory cytokines, characteristic of M1 activation, but also express high levels of the anti-inflammatory cytokine IL-10.

Finally, the M2c macrophage state is stimulated by IL-10 and is sometimes referred to as a regulatory macrophage. M2c macrophages have anti-inflammatory activity that plays a role in the phagocytosis of cellular debris without the classical pro-inflammatory response. These cells express TGFβ and high IL-10 as well as matrix proteins. Plunkett et al. reported that IL-10 mediated anti-inflammatory responses including decreasing glial activation and production of pro-inflammatory cytokines.

Several approaches have been proposed to modulate microglial activation as potential targets for treatment of neurodegenerative processes. It has been suggested that use of anti-inflammatory medications, such as non-steroidal anti-inflammatory drugs (NSAIDs), to halt the progression of neurodegenerative processes could be suppressing both pro-inflammatory and anti-inflammatory activation by endogenous molecules, inactivating the beneficial effect of M2 microglial functions and endogenous mechanisms of plaque clearance.

Prior research has focused primarily on two areas: anti-inflammatory agents to temper toxic effect of pro-inflammatory cytokines; and converting microglia from M1 state to an M2 state in which the toxic effects are reduced and their phagocytic activity is enhanced. Multiple anti-inflammatory agents have been tested, but have shown little or no efficacy in the conversion of microglia from M1 state to M2 state.

Accordingly, anti-inflammatory treatments of neuro inflammatory conditions by modulating conversion of microglia from M1 state to M2 state are needed.

### SUMMARY

In certain embodiments, the invention relates to a method of treating or slowing the progression of a disease or condition in a subject in need thereof comprising co-administering a first compound and a second compound,

wherein

the disease or condition is a neuron inflammation condition; and

the first compound and the second compound are independently

(a) a compound having formula (I):

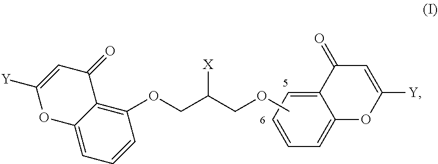

wherein

X is halide, hydroxyl, or OCO(C₁₋₈alkyl);

Y is CO₂R¹ or CH₂OR²;

R¹ is Li, Na, K, H, C₁₋₄alkyl, or —CH₂CO₂(C₁₋₅alkyl); and

R² is H or —C(O)(C₁₋₄alkyl),

or pharmaceutically acceptable salts thereof; or

(b) selected from bitolterol, fenoterol, isoprenaline, levosalbutamol, orciprenaline, pirbuterol, procaterol, ritodrine, salbutamol, terbutaline, arformoterol, bambuterol, clenbuterol, formoterol, salmeterol, abediterol, carmoterol, indacaterol, olodaterol, vilanterol, nedocromil, ketotifen, olopatadine, omalizumab, quercetine, mepolizumab, azelastine, and methylxanthines pemirolast, olopataidne, alfatoxin G₁, alfatoxin B₁, alfatoxin M₁, deoxynivalenol, zearalenone, ochratoxin A, fumonisin B₁, hydrolyzed fumonisin B₁, patulin, and ergotamine; or

(c) edaravone or riluzole; or

(d) selected from.

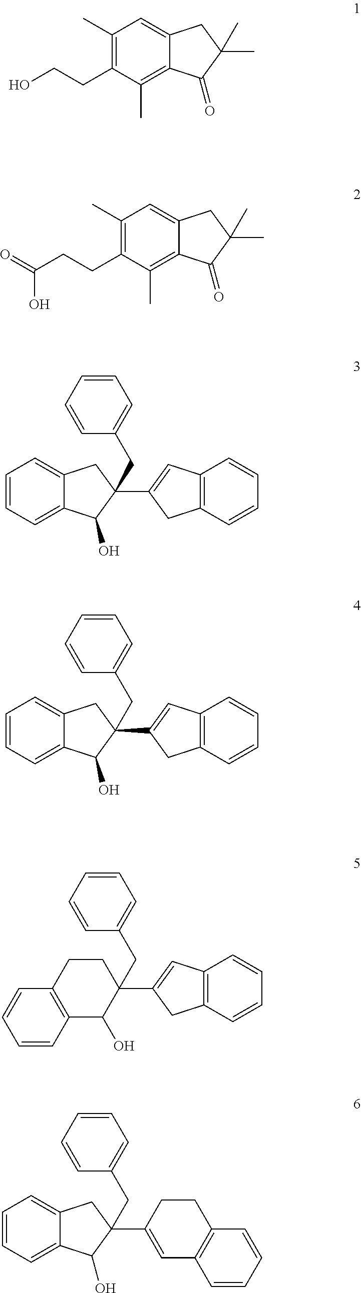 [SMILES: Cc1cc2c(c(C)c1CCO)C(=O)C(C)(C)C2 ; Cc1cc2c(c(C)c1CCC(=O)O)C(=O)C(C)(C)C2 ; O[C@H]1c2ccccc2C[C@]1(Cc1ccccc1)C1=Cc2ccccc2C1 ; O[C@H]1c2ccccc2C[C@@]1(Cc1ccccc1)C1=Cc2ccccc2C1 ; OC1c2ccccc2CCC1(Cc1ccccc1)C1=Cc2ccccc2C1 ; OC1c2ccccc2CC1(Cc1ccccc1)C1=Cc2ccccc2CC1]

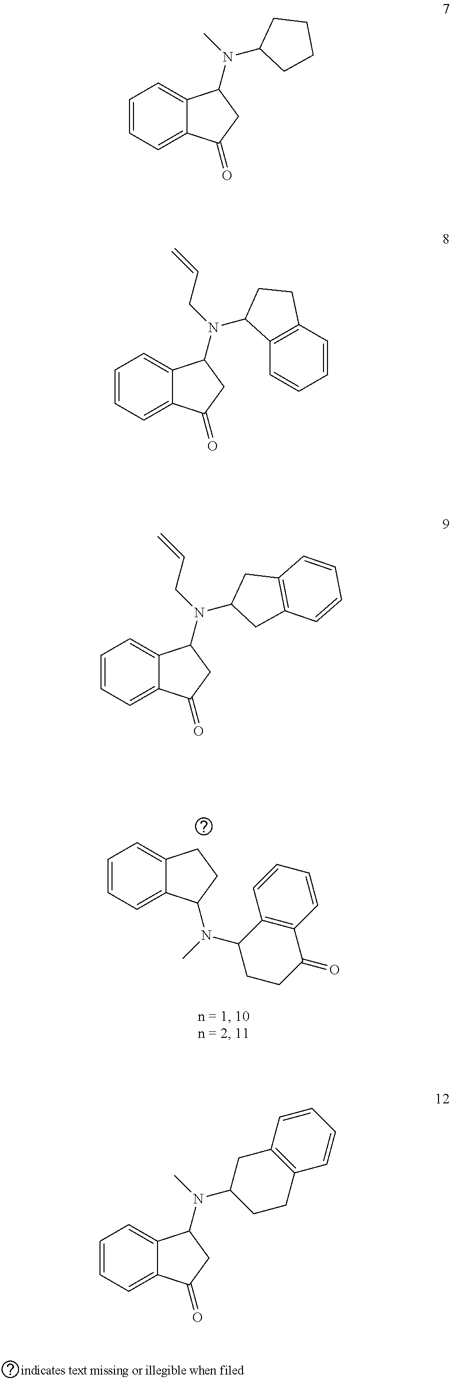 [SMILES: CN(C1CCCC1)C1CC(=O)c2ccccc21 ; C=CCN(C1CCc2ccccc21)C1CC(=O)c2ccccc21 ; C=CCN(C1Cc2ccccc2C1)C1CC(=O)c2ccccc21 ; CN(C1CCc2ccccc21)C1CCC(=O)c2ccccc21 ; CN(C1CCc2ccccc2C1)C1CC(=O)c2ccccc21]

or pharmaceutically acceptable salts thereof; or

(e) a non-steroidal anti-inflammatory drug (NSAID); or

(f) an anti-inflammatory peptide; and

the first compound and the second compound, taken together, are therapeutically effective.

In certain embodiments, the invention relates to co-administration of cromolyn or a salt or ester thereof and edaravone for the treatment of ALS.

In certain embodiments, the invention relates to co-administration of cromolyn or a salt or ester thereof and riluzole for the treatment of ALS.

### BRIEF DESCRIPTION OF THE FIGURES

FIG. 1A-FIG. 1C are graphs showing that cromolyn sodium treatment does not alter body weight of TgSOD1 mice. FIG. 1A depicts a two-way ANOVA and Tukey's multiple comparison test revealed a significant improvement in body weight in the TgSOD1-Cromolyn group compared to TgSOD1-Vehicle group at P130 only. There was also a significant decrease in body weight in the TgSOD1-Vehicle group compared to WtSOD1-Vehicle and WtSOD1-Cromolyn at P100, P110, P120, P130, P140, and P150. There was also a significant decrease in body weight in the TgSOD1-Cromolyn group compared to WtSOD1-Cromolyn group at P100, P110, P120, P130, P140, and P150. There was a significant difference in body weight between TgSOD1-Cromolyn group and WtSOD1-Vehicle group at P120, P130, and P140 only. FIG. 1B depicts, in female mice, two-way ANOVA and Tukey's multiple comparison test revealed a significant decrease in body weight in the TgSOD1-Vehicle group compared to wild-type groups at P120 and P130 (FIG. 1b). There was also a significant decrease in body weight in the TgSOD1-Cromolyn group compared to wild-type groups at P130, P140, and P150. FIG. 1C depicts, in male mice, two-way ANOVA and Tukey's multiple comparison test revealed a significant decrease in body weight in the TgSOD1-Vehicle group compared to wild-type groups at P90, P100, P110, P120, P130, and P140. There was also a significant decrease in body weight in the TgSOD1-Cromolyn group compared to wild-type groups at P90, P100, P110, P120, and P130. Female mice: WtSOD1-Vehicle (n=19; light grey), WtSOD1-Cromolyn (n=17; dark grey), TgSOD1-Vehicle (n=19; black), and TgSOD1-Cromolyn (n=17; red). Male mice: WtSOD1-Vehicle (n=18; light grey), WtSOD1-Cromolyn (n=21; dark grey), TgSOD1-Vehicle (n=21; black), TgSOD1-Cromolyn (n=17; red). * denotes differences between TgSOD1-Vehicle and Tg-SOD1-Cromolyn; \hat{ } denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; **p<0.01; *** p<0.001; *** p<0.0001, the same statistical significance is associated with each symbol. Data are presented as median and interquartile ranges.

FIG. 2A-FIG. 2C are graphs showing that cromolyn sodium treatment improved neurological score and delayed disease onset in TgSOD1 mice. FIG. 2A shows a two-way ANOVA demonstrated and Tukey's post-hoc analysis revealed a significant increase in neurological score in the TgSOD1-Vehicle treated group compared to TgSOD1-Cromolyn group at P90, P100, P110, P130, and P140. FIG. 2B shows, in female mice, two-way ANOVA and Tukey's post-hoc analysis revealed a significant increase in neurological score in the female TgSOD1-Vehicle treated group compared to TgSOD1-Cromolyn group at P90, P100, P120, P130, and P140. FIG. 2C shows, in male mice, two-way ANOVA and Tukey's post-hoc analysis revealed a significant increase in neurological score in male TgSOD1-Vehicle treated group compared to TgSOD1-Cromolyn group at P90, P100, and P110. Number of mice per groups, females Female mice: WtSOD1-Vehicle (n=19; light grey), WtSOD1-Cromolyn (n=17; dark grey), TgSOD1-Vehicle (n=19; black), and TgSOD1-Cromolyn (n=17; red). Male mice: WtSOD1-Vehicle (n=18; light grey), WtSOD1-Cromolyn (n=21; dark grey), TgSOD1-Vehicle (n=21; black), TgSOD1-Cromolyn (n=17; red). * denotes differences between TgSOD1-Vehicle and Tg-SOD1-Cromolyn; \hat{ } denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; ** p<0.01; *** p<0.001; *** p<0.0001, the same statistical significance is associated with each symbol. Data are presented as median and interquartile ranges.

FIG. 3A-FIG. 3C are graphs showing the effect of cromolyn sodium treatment on performance on the PAGE task in TgSOD1 mice. FIG. 3A shows a two-way ANOVA and Tukey's post-hoc analysis revealed a significant improvement in PaGE performance in TgSOD1-Cromolyn compared to TgSOD1-Vehicle group at P120 and P140. There was a significant decrease in PaGE in the TgSOD1-Vehicle group at P80, P100, P120, and P140 compared to WtSOD1-Vehicle and WtSOD1-Cromolyn groups. In addition, there was a significant decrease in PaGE in the TgSOD1-Cromolyn group compared to both wild-type groups at P100 and P120. FIG. 3B shows, in female mice, two-way ANOVA and Tukey's post-hoc analysis revealed a significant decrease in PaGE in the TgSOD1-Vehicle group at P120 and P140 compared to WtSOD1-Vehicle and WtSOD1-Cromolyn groups. In addition, there was a significant decrease in PaGE in the TgSOD1-Cromolyn group compared to both wild-type groups at P100 and P120. Importantly, there was a significant difference between the two transgenic groups at P100 with a worsening in the cromolyn treated female group, and an improvement in PaGE performance at P140 in the treated group. FIG. 3C shows, in male mice, two-way ANOVA and Tukey's post-hoc analysis revealed a significant decrease in PaGE in the TgSOD1-Vehicle group compared to both wild-type groups at P80, P100, and P120. There was also a significant decrease in PaGE in the TgSOD1-Cromolyn group compared to both wild-type groups at P100 and P120. Importantly, there was a significant improvement in PaGE at P120 between the two male transgenic groups. Female mice: WtSOD1-Vehicle (n=19; light grey), WtSOD1-Cromolyn (n=17; dark grey), TgSOD1-Vehicle (n=19; black), and TgSOD1-Cromolyn (n=17; red). Male mice: WtSOD1-Vehicle (n=18; light grey), WtSOD1-Cromolyn (n=21; dark grey), TgSOD1-Vehicle (n=21; black), TgSOD1-Cromolyn (n=17; red). Data are presented as median and interquartile ranges. * denotes differences between TgSOD1-Vehicle and Tg-SOD1-Cromolyn; \hat{ } denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; ** p<0.01; *** p<0.001; *** p<0.0001, the same statistical significance is associated with each symbol. Data are presented as median and interquartile ranges.

FIG. 4A-FIG. 4C show that cromolyn sodium did not alter performance on the rotarod. FIG. 4A shows a two-way ANOVA and Tukey's post-hoc analysis revealed no difference in rotarod performance between the TgSOD1-Vehicle and TgSOD1-Cromolyn mice. There was a significant difference between TgSOD1-Vehicle with both WtSOD1-Vehicle and WtSOD1-Cromolyn at P70, P90 and P120. Similarly, post-hoc analysis revealed a significant decrease in rotarod performance between TgSOD1-Cromolyn group compared to WtSOD1-Vehicle and WtSOD1-Cromolyn at all time points. FIG. 4B shows, in female mice, two-way ANOVA and Tukey's post-hoc analysis revealed a significant difference between TgSOD1-Vehicle with both WtSOD1-Vehicle and WtSOD1-Cromolyn at P70, P90 and P120. Similarly, post-hoc analysis revealed a significant decrease in rotarod performance between TgSOD1-Cromolyn group compared to WtSOD1-Vehicle and WtSOD1-Cromolyn at 681 all time points. FIG. 4C shows, in male mice, two-way ANOVA and Tukey's post-hoc analysis revealed a significant difference between TgSOD1-Vehicle with both WtSOD1-Vehicle and WtSOD1-Cromolyn at P70, P90 and P120 in male treated mice. Similarly, post-hoc analysis revealed a significant decrease in rotarod performance between male TgSOD1-Cromolyn group compared to WtSOD1-Vehicle and WtSOD1-Cromolyn at all time points. Female mice: WtSOD1-Vehicle (n=19; light grey), WtSOD1-Cromolyn (n=17; dark grey), TgSOD1-Vehicle (n=19; black), and TgSOD1-Cromolyn (n=17; red). Male mice: WtSOD1-Vehicle (n=18; light grey), WtSOD1-Cromolyn (n=21; dark grey), TgSOD1-Vehicle (n=21; black), TgSOD1-Cromolyn (n=17; red). * denotes differences between TgSOD1-Vehicle and Tg-SOD1-Cromolyn; \hat{ } denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; **p<0.01; *** p<0.001; *** p<0.0001, the same statistical significance is associated with each symbol. Data are presented as median and interquartile ranges.

FIG. 5A-FIG. 5C show that cromolyn sodium did not alter gait performance. FIG. 5A shows a two-way ANOVA and Tukey's post-hoc analysis revealed no significant difference in stride length between TgSOD1-cromolyn and TgSOD1-Vehicle groups. There was a significant decrease in stride length in TgSOD1-Vehicle compared with both wild-type groups at P120. Similarly, post-hoc analysis revealed a significant decrease in stride length in TgSOD1-Cromolyn group compared wild-type mice at P120 suggesting that cromolyn treatment had no effect on stride length. FIG. 5B shows, in female mice, two-way ANOVA and Tukey's post-hoc analysis revealed a significant decrease in stride length in TgSOD1-Vehicle and TgSOD1-Cromolyn treated female mice compared with both wild-type groups at P120 (FIG. 5b). FIG. 5C shows, in male mice, two-way ANOVA and Tukey's post-hoc analysis revealed a significant decrease in stride length in male TgSOD1-Vehicle and TgSOD1-Cromolyn treated mice compared with both wild-type groups at P120.

FIG. 6A-FIG. 6C show that cromolyn sodium did not stride width. FIG. 6A shows a two-way ANOVA and Tukey's post-hoc analysis revealed a significant increase in stride width at P120 in TgSOD1-Vehicle group compared to WtSOD1-Vehicle. FIG. 6B shows, in female mice, two-way ANOVA revealed a significant effect on age on stride width. FIG. 6C shows, in male mice, two-way ANOVA and Tukey's analysis revealed a significant increase in stride width in the TgSOD1-Vehicle treated mice compared to both wild-type groups. Female mice: WtSOD1-Vehicle (n=19; light grey), WtSOD1-Cromolyn (n=17; dark grey), TgSOD1-Vehicle (n=19; black), and TgSOD1-Cromolyn (n=17; red). Male mice: WtSOD1-Vehicle (n=18; light grey), WtSOD1-Cromolyn (n=21; dark grey), TgSOD1-Vehicle (n=21; black), TgSOD1-Cromolyn (n=17; red). * denotes differences between TgSOD1-Vehicle and Tg-SOD1-Cromolyn; \hat{ } denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; **p<0.01; *** p<0.001; *** p<0.0001, the same statistical significance is associated with each symbol. Data are presented as median and interquartile ranges.

FIG. 7A-FIG. 7C show cromolyn sodium treatment delayed the age at paresis onset in TgSOD1 mice. FIG. 7A shows that there was a significant effect of cromolyn treatment on the onset of motor symptoms as measured by age at paresis onset (Mantel-Cox test), with a median age of onset of 99 days for TgSOD1-Vehicle group and 107 days for the TgSOD1-Cromolyn group. FIG. 7B shows, in female mice there was a significant delay in the onset of motor symptoms following cromolyn treatment (Mantel-Cox test). FIG. 7C shows, in male mice, cromolyn treatment significantly delayed the onset of motor symptoms (Mantel-Cox test). Female mice: WtSOD1-Vehicle (n=19; light grey), WtSOD1-Cromolyn (n=17; dark grey), TgSOD1-Vehicle (n=19; black), and TgSOD1-Cromolyn (n=17; red). Male mice: WtSOD1-Vehicle (n=18; light grey), WtSOD1-Cromolyn (n=21; dark grey), TgSOD1-Vehicle (n=21; black), TgSOD1-Cromolyn (n=17; red). * denotes differences between TgSOD1-Vehicle and Tg-SOD1-Cromolyn; \hat{ } denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; **p<0.01; *** p<0.001; *** p<0.0001, the same statistical significance is associated with each symbol. Data are presented as median and interquartile ranges.

FIG. 8A-FIG. 8C depict that cromolyn sodium increased survival in female TgSOD1 mice. FIG. 8A shows that cromolyn treatment did not have a significant effect on survival in cromolyn treated mice (Mantel-Cox). FIG. 8B shows that there was a significant effect of treatment on survival in female mice only (Mantel-Cox test). FIG. 8C shows that there was no effect of treatment on male mice. Data are presented as median and interquartile ranges. Female mice: WtSOD1-Vehicle (n=19; light grey), WtSOD1-Cromolyn (n=17; dark grey), TgSOD1-Vehicle (n=19; black), and TgSOD1-Cromolyn (n=17; red). Male mice: WtSOD1-Vehicle (n=18; light grey), WtSOD1-Cromolyn (n=21; dark grey), TgSOD1-Vehicle (n=21; black), TgSOD1-Cromolyn (n=17; red). * denotes differences between TgSOD1-Vehicle and Tg-SOD1-Cromolyn; \hat{ } denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; **p<0.01; *** p<0.001; *** p<0.0001, the same statistical significance is associated with each symbol. Data are presented as median and interquartile ranges.

FIG. 9A-FIG. 9B show that cromolyn treatment is neuroprotective and increases survival of lumbar spinal cord motor neurons in TgSOD1 mice. FIG. 9A shows representative images of lumbar spinal cord motor neurons visualized by H&E staining. FIG. 9B shows a one-way ANOVA and Dunn's multiple comparisons test demonstrated that motor neuron survival was significantly increased in the TgSOD1-Cromolyn group compared to TgSOD1-Vehicle group. There was a significant decrease in motor neuron counts in the TgSOD1-Vehicle group compared to WtSOD1-Vehicle and WtSOD1-Cromolyn groups. There was also a decrease in motor neuron counts in TgSOD1-Cromolyn group compared to both wild-type groups. WtSOD1-Vehicle (n=19; light grey), WtSOD1-Cromolyn 755 (n=17; dark grey), TgSOD1-Vehicle (n=19; black), and TgSOD1-Cromolyn (n=17; red). * denotes differences between TgSOD1-Vehicle and Tg-SOD1-Cromolyn; \hat{ } denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; ** p<0.01; *** p<0.001; *** p<0.0001, the same statistical significance is associated with each symbol. Data are presented as median and interquartile ranges.

FIG. 10A-FIG. 10B show cromolyn treatment does not alter microgliosis in the spinal cord of TgSOD1 mice. FIG. 10A shows microglia of the lumbar spinal cord were visualized using the Iba1-specific antibody and DAB staining. FIG. 10B shows quantifications of the percentage of Iba1-positive cell area revealed no difference in the percentage of Iba1-positive cell area in TgSOD1-Cromolyn mice compared to TgSOD1-Vehicle. There was a significant increase in the percentage of Iba1-positive cell area in the spinal cord of both vehicle and TgSOD1-Cromolyn compared to both wild-type groups as demonstrated by one-way ANOVA and Tukey's post-hoc analysis. WtSOD1-Vehicle (n=19; light grey), WtSOD1-Cromolyn (n=17; dark grey), TgSOD1-Vehicle (n=19; black), and TgSOD1-Cromolyn (n=17; red). * denotes differences between TgSOD1-Vehicle and Tg-SOD1-Cromolyn; \hat{ } denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; ** p<0.01; *** p<0.001; *** p<0.0001, the same statistical significance is associated with each symbol. Data are presented as median and interquartile ranges.

FIG. 11A-FIG. 11E are graphs showing that cromolyn treatment decreased the levels of pro-inflammatory cytokines/chemokines in the spinal cord of TgSOD1 mice. FIG. 11A: IL-1b. FIG. 11B: IL-5. FIG. 11C: IL-6. FIG. 11D: CXCL1. FIG. 11E: TNFα. One-way ANOVA and post-hoc analysis revealed that cromolyn treatment significantly decreased CXCL1 (FIG. 11D) and TNFα (FIG. 11E) levels in the TgSOD1-Cromolyn group compared to TgSOD1-Vehicle group. There was a significant difference in the levels of IL-1b (FIG. 11A), IL-5 (FIG. 11B), IL-6 (FIG. 11C), CXCL1 (FIG. 11D), and TNFα (FIG. 11E) in the spinal cord of both TgSOD1-Vehicle and TgSOD1-Cromolyn groups compared to both wild-type groups. While there was a significant increase in IL-1b (FIG. 11A), CXCL1 (FIG. 11D), and TNFα (FIG. 11E), there was a significant decrease in IL-5 (FIG. 11B) and IL-6 (FIG. 11C) levels between Tg and Wt groups. WtSOD1-Vehicle (n=15; light grey), WtSOD1-Cromolyn (n=19; dark grey), TgSOD1-Vehicle (n=17; black), and TgSOD1-Cromolyn (n=17; red). * denotes differences between TgSOD1-Vehicle and Tg-SOD1-Cromolyn; A denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; ** p<0.01; *** p<0.001; *** p<0.0001, the same statistical significance is associated with each symbol. Data are presented as median and interquartile ranges.

FIG. 12A-FIG. 12G are graphs showing that cromolyn treatment decreased the levels of pro-inflammatory cytokines/c 792 hemokines in plasma of TgSOD1 mice. FIG. 12B, FIG. 12D, and FIG. 12E show a one-way ANOVA and post-hoc analysis revealed a significant decrease in IL-2 (FIG. 12B), IL-6 (FIG. 12D), and IL-10 (FIG. 12E) levels in TgSOD1-Cromolyn group compared to TgSOD1-Vehicle group. There was a significant difference in IL-2 (FIG. 12B), IL-6 (FIG. 12D), and IL-10 (FIG. 12E), and TNFα (FIG. 12G) levels in the plasma of TgSOD1-Vehicle compared to both WtSOD1-Vehicle and WtSOD1-Cromolyn groups. One-way ANOVA demonstrated a trend towards an increase in CXCL1 (FIG. 12F) levels in TgSOD1-Vehicle mice compared to WtSOD1-Vehicle group and a trend towards an increase compared to WtSOD1-Cromolyn group. There was no statistically significant difference between IL-10 (FIG. 12A) and IL-5 (FIG. 12C) levels between groups. Lastly, there was a trend towards a decrease in TNFα levels (FIG. 12G) in the TgSOD1-Cromolyn mice compared to the TgSOD1-Vehicle group. WtSOD1-Vehicle (n=11; light grey), WtSOD1-Cromolyn (n=11; dark grey), TgSOD1-Vehicle (n=9; black), and TgSOD1-Cromolyn (n=9; red). * denotes differences between TgSOD1-Vehicle and Tg-SOD1-Cromolyn; \hat{ } denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; ** p<0.01; *** p<0.001; *** p<0.0001, the same statistical significance is associated with each symbol. Data are presented as median and interquartile ranges.

FIG. 13A-FIG. 13E shows that cromolyn treatment increased GPR35 levels in spinal cord. FIG. 13A shows representative immunoblots of GPR35 and b-actin from spinal cord samples. FIG. 13B shows a one-way ANOVA and Tukey's post-hoc analysis of spinal cord western blots revealed that there was a trend towards an increase in GPR35 levels in TgSOD1-Cromolyn compared to the TgSOD1-Vehicle group. There was a significant decrease in GPR35 levels in TgSOD1-Vehicle group compared to WtSOD1-Vehicle and WtSOD1-Cromolyn. There was no significant difference in GPR35 levels between the TgSOD1-Cromolyn group and either of the wild-type groups. FIG. 13C shows representative immunofluorescence images of GPR35 (red), NeuN (green), and merged images from the lumbar spinal cord. GPR35 is co-localized with the neuronal marker, NeuN. FIG. 13D shows a one-way ANOVA and Tukey's post-hoc analysis revealed a significant increase in GPR35 levels in TgSOD1-Cromolyn group compared to TgSOD1-Vehicle. FIG. 13E shows a one-way ANOVA and post-hoc analysis revealed a significant increase in neuronal GPR35 levels in the TgSOD1-Cromolyn group compared to TgSOD1-Vehicle. For western blots WtSOD1-Vehicle (n=21; light grey), WtSOD1-Cromolyn (n=19; dark grey), TgSOD1-Vehicle (n=18; black), and TgSOD1-Cromolyn (n=17; red). For Immunofluorescence WtSOD1-Vehicle (n=6; light grey), WtSOD1-Cromolyn (n=6; dark grey), TgSOD1-Vehicle (n=6; black), and TgSOD1-Cromolyn (n=6; red). * denotes differences between TgSOD1-Vehicle and Tg-SOD1-Cromolyn; \hat{ } denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; ** p<0.01; *** p<0.001; *** p<0.0001, the same statistical significance is associated with each symbol. Data are presented as median and interquartile ranges.

FIG. 14A-FIG. 14B shows that cromolyn treatment does not alter MCP-1 levels in the spinal cord or plasma of TgSOD1 mice. FIG. 14A shows a one-way ANOVA and post-hoc analysis revealed a significant increase in MCP-1 levels in the spinal cord of TgSOD1-Vehicle mice compared to both WtSOD1-Vehicle and WtSOD1-Cromolyn groups. However, there was no effect of cromolyn treatment on MCP-1 levels in the spinal cord of TgSOD1-Cromolyn compared to TgSOD1-Vehicle. FIG. 14B shows a one-way ANOVA and post-hoc analysis revealed that MCP-1 levels were not altered in the plasma in any of the groups. For spinal cord WtSOD1-Vehicle (n=15; light grey), WtSOD1-Cromolyn (n=19; dark grey), TgSOD1-Vehicle (n=17; black), and TgSOD1-Cromolyn (n=17; red). For plasma WtSOD1-Vehicle (n=11; light grey), WtSOD1-Cromolyn (n=11; dark grey), TgSOD1-Vehicle (n=9; black), and TgSOD1-Cromolyn (n=9; red). * denotes differences between TgSOD1-Vehicle and Tg-SOD1-Cromolyn; \hat{ } denotes differences between TgSOD1-Vehicle and WtSOD1-Vehicle; #denotes differences between TgSOD1-Vehicle and WtSOD1-Cromolyn; @ denotes differences between TgSOD1-Cromolyn and WtSOD1-Vehicle; % denotes differences between TgSOD1-Cromolyn and WtSOD1-Cromolyn. * p<0.05; **p<0.01; *** p<0.001; *** p<0.0001, the same statistical significance is associated with each symbol. Data are presented as median and interquartile ranges.

### DETAILED DESCRIPTION

### Overview

The present disclosure demonstrates a potentially new mechanism of action for cromolyn, an FDA-approved drug used in the treatment of asthma, in combination with other therapeutics. As described herein, cromolyn displays a significant ability to modulate immune microglia activation from the M1 aggressive state to the M2 phagocytic state and is expected to slow down or halt motor neuron degeneration. M2-state microglia devour excess inflammatory cytokines and, by so doing, prevent the spread of synaptic and neural damage. Cromolyn is available in subcutaneous injectable form (Iradica-Q), providing improved bioavailability. Additional studies have shown that cromolyn binds to beta-amyloid and alpha-synuclein peptides, inhibiting their polymerization to higher order aggregates. Aggregation of these peptides was observed in familial ALS (FALS) subjects, where the aggregation is caused by the mutation of the SOD1 protein. Cromolyn may act as an inhibitor for the aggregation of SOD1 monomers. In vitro studies showed that cromolyn inhibits the SOD1 gene. Furthermore, studies have shown that cromolyn penetrates the blood-brain barrier, both in an animal model and in human pharmacokinetic studies. Pharmacokinetics of cromolyn in both the blood plasma and CSF of healthy volunteers and in subjects with Alzheimer's disease has been studied.

Plasma bioavailability following cromolyn subcutaneous injection translates to concentrations that ameliorate the neuro inflammation associated with cytokines, free radicals, and toxins in the brain, sufficient to interfere with SOD1 accumulation and precipitation.

Further studies in an animal model using SOD1 transgenic mice (mice developing SOD1 burden in the brain) provided statistically significant evidence of the benefit of intraperitoneally (IP) administered cromolyn. These results indicate that Iradica-Q treatment has a potential to slow down the decline in behavior caused by brain SOD Iburden in a transgenic animal model of ALS.

As described herein, cromolyn sodium treatment delayed disease onset and progression, reduced motor deficits in the Paw Grip Endurance (PaGE) task, and improved survival (female mice only) in the SOD1^{G93A} mouse model. Furthermore, cromolyn treatment significantly spared lumbar spinal cord motor neurons and reduced pro-inflammatory cytokine/chemokine levels in the spinal cord and plasma of TgSOD1 mice. Lastly, cromolyn treatment led to an increase in neuronal GPR35 levels. Together, these findings suggest that cromolyn may regulate the immune response in TgSOD1 mice via activation of GPR35.

In certain embodiments, cromolyn inhibits mast cell degranulation and is used to treat asthma, allergic rhinitis, mastocytosis, and conjunctivitis. In animal models, cromolyn treatment attenuates activation and degranulation of mast cells, reduces histamine expression and infiltration of macrophages. Furthermore, cromolyn decreases the expression of pro-inflammatory chemokines and cytokines such as IL-1β, IL-6, TNFα, CCL3 and MCP1. In certain embodiments, cromolyn decreases Aβ aggregation in young Tg2576 AD 333 mice with minimal amyloid deposition. Cromolyn also significantly impacts brain Aβ levels in APP^{Swedish}-expressing Tg2576 mice. These observed effects of cromolyn on recruitment of microglia to plaques and enhanced microglial uptake of Aβ suggest that cromolyn may convert microglial activation state from one favoring neuroinflammation to one promoting phagocytosis.

Microgliosis is significantly increased in the spinal cord of TgSOD1 compared to WtSOD1 mice; however, as described herein, there was no difference between vehicle and cromolyn treated TgSOD1 mice. However, the alterations in pro-inflammatory cytokines and chemokines in response to cromolyn may induce a shift in microglial activation states from pro- to anti-inflammatory. To further delineate the effects of cromolyn treatment on inflammation, alterations in cytokines and chemokines in the spinal cord were measured. Pro-inflammatory cytokines, IL-1β and TNFα, and the chemokine CXCL1, were significantly increased in the spinal cord, while the cytokines IL-5 and IL-6 were decreased in the spinal cord of TgSOD1 mice compared to WtSOD1 mice. Importantly, cromolyn sodium treatment significantly decreased CXCL1 and TNFα levels in the spinal cord of TgSOD1 mice.

CXCL1 is a chemotactic cytokine responsible for mediating migration of neutrophils to the sight of inflammation; in patients with ALS, CXCL1 levels are significantly increased. Specifically, CXCL1 levels are increased in monocytes isolated from ALS patients and in ALS patient-derived fibroblasts. In certain embodiments, the disclosure relates to the discovery that CXCL1 levels are increased in the spinal cord of TgSOD1 mice similar to what has been previously reported in ALS patients. Given that CXCL1 has been shown to contribute to the transendothelial migration of monocytes from blood to the brain in AD patients, it may contribute to peripheral nerve invasion by macrophages in ALS patients. Therefore, lowering CXCL1 expression using cromolyn could be highly beneficial for decreasing the inflammatory response in ALS patients.

In certain embodiments, cromolyn sodium treatment also significantly decreased the levels of TNFα in the spinal cord of TgSOD1 mice. Although astrocytes and neurons are able to produce TNFα, it is assumed that microglia are the major source of TNFα release during neuroinflammation. TNFα has been shown to potentiate AMPAR-mediated excitotoxicity on lumbar spinal cord motor neurons by decreasing GLT-1 expression, and by inducing a rapid membrane insertion of Ca²⁺ permeable-AMPARs. Therefore, in certain embodiments, cromolyn treatment could provide some of its neuroprotective effects by decreasing AMPA-mediated excitotoxicity. In certain embodiments, an increase in GPR35, the endogenous target receptor for cromolyn sodium, was observed following treatment.

Although GPR35 is predominantly expressed in immune cells it has been implicated in cardiovascular, inflammatory, and neurological disease. GPR35 contributes to the anti-inflammatory effects of aspirin and the anti-allergic effects of cromolyn. In addition to its role in inflammation, activation of GPR35 in peripheral nervous system neurons leads to dampening of mechanisms involved in synaptic transmission. Specifically, activation of GPR35 in sympathetic neurons resulted in the inhibition of voltage-gated Ca²⁺ channels and forskolin-induced cyclic-AMP (cAMP) production in dorsal root ganglion. Additionally, in the CNS, GPR35 activation suppressed neuronal firing in the CA1 region of the hippocampus. Together, these findings suggest that GPR35 activity alters neuronal excitability and synaptic transmission and therefore, in addition to dampening the inflammatory response, activation of GPR35 may also provide additional therapeutic benefit by preventing excitotoxicity in ALS.

Pro-inflammatory cytokines, such as IL-1β, TNF-α, IFN-γ, IL-6, and IL-8 have been reported to be elevated in plasma or serum samples of ALS patients, with levels increasing with disease progression. Furthermore, peripheral blood inflammatory cytokines have been suggested as diagnostic biomarkers for ALS. Therefore, in certain embodiments, the disclosure relates to the effects of cromolyn sodium treatment in the periphery in order to identify a pharmacodynamic biomarker for the treatment. Cytokines IL-2, IL-6, and IL-10 were increased in the plasma of TgSOD1 mice compared to WtSOD1 mice. Furthermore, a significant increase in TNFα and CXCL1 levels were observed in the plasma of TgSOD1 mice compared to wild-type mice, similar to the findings in the spinal cord. Cromolyn treatment resulted in significantly decreased levels of IL-2, IL-6, and IL-10, as well as a trend towards decreased TNFα levels in the plasma, suggesting that cromolyn treatment reduces inflammation in the peripheral blood of TgSOD1 mice. While IL-2 and TNFα are considered to be pro-inflammatory cytokines, IL-6 exhibits both pro- and anti-inflammatory properties. Interestingly, IL-10 has been shown to inhibit inflammatory response by metabolic reprogramming of macrophages. In certain embodiments, cromolyn treatment led to a decrease in the levels of peripheral blood inflammatory cytokines, which are potential diagnostic biomarkers for ALS. Although MCP-1 levels were increased in the spinal cord but not plasma of TgSOD1 mice and in the cerebrospinal fluid of ALS patients, cromolyn treatment did not impact 407 MCP-1 levels in the spinal cord or plasma of TgSOD1 mice.

In certain embodiments, the disclosure relates to the discovery that cromolyn sodium treatment (6.3 mg/kg) significantly improved performance in the PaGE task and delayed the onset of disease in both male and female mice. However, there was a female specific effect on improved survival. Studies suggest distinct disease progression and greater therapeutic improvements in transgenic female SOD1^{G93A} mice compared to male mice. Specifically, female TgSOD1^{G93A} mice were shown to exhibit prolonged survival compared to male cohorts. In certain embodiments, cromolyn sodium demonstrates a greater neuroprotective effect in the female TgSOD1 mice. While not wishing to be bound by any particular theory, an alternative explanation for these findings is the possible interaction of cromolyn with the female sex hormone receptors.

Together, these findings demonstrate that cromolyn sodium treatment delays disease onset and progression, reduces motor deficits (PaGE), and improves survival (female mice only) in the SOD1^{G93A} mouse model. Furthermore, cromolyn treatment significantly increased the survival of lumbar spinal cord motor neurons. While cromolyn treatment did not impact microgliosis, it reduced pro-inflammatory cytokine and chemokine levels in the spinal cord and plasma of SOD1^{G93A} mice, suggesting the cromolyn alters microglial activation state (towards anti-inflammatory). Cromolyn treatment also increased GPR35 levels suggesting that some of cromolyn-mediated effects may be through GPR35 activation. Therefore, cromolyn in combination with other agents are useful for the treatment of ALS.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g., “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁₋₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y},” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. The terms “C_{2-y} alkenyl” and “C_{2-y} alkynyl” refer to substituted or unsubstituted unsaturated aliphatic groups analogous in length and possible substitution to the alkyls described above, but that contain at least one double or triple bond respectively.

All of the above, and any other publications, patents and published patent applications referred to in this application are specifically incorporated by reference herein. In case of conflict, the present specification, including its specific definitions, will control.

The term “patient” or “subject” refers to a mammal in need of a particular treatment. In a preferred embodiment, a patient is a primate, canine, feline, or equine. In another preferred embodiment, a patient is a human.

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration of the disease state, and remission (whether partial or total), whether detectable or undetectable. “Treatment” can also mean prolonging survival as compared to expected survival if not receiving treatment. In addition, this term includes supportive treatment, that is, treatment employed to supplement another specific therapy directed toward the improvement of the associated disease, pathological condition, or disorder.

With regard to administering the compound, the term “administering” refers to any method of providing a composition and/or pharmaceutical composition thereof to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intraarterial administration, intramuscular administration, subcutaneous administration, intravitreous administration, and the like. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The terms “co-administration” and “co-administering” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent at the same time.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

### Combination Therapy

One aspect of the present invention relates to combination therapy. This type of therapy is advantageous because the co-administration of active ingredients achieves a therapeutic effect that is greater than the therapeutic effect achieved by administration of only a single therapeutic agent.

In certain embodiments, the co-administration of two or more therapeutic agents achieves a therapeutic effect that is greater than the therapeutic effect achieved by administration of only a single therapeutic agent. In this regard, the combination therapies are efficacious. The therapeutic effect of one therapeutic agent is augmented by the co-administration of another therapeutic agent.

In certain embodiments, the co-administration of two or more therapeutic agents achieves a therapeutic effect that is equal to about the sum of the therapeutic effects achieved by administration of each single therapeutic agent. In these embodiments, the combination therapies are said to be “additive.”

In certain embodiments, the co-administration of two or more therapeutic agents achieves a synergistic effect, i.e., a therapeutic effect that is greater than the sum of the therapeutic effects of the individual components of the combination.

The active ingredients that comprise a combination therapy may be administered together via a single dosage form or by separate administration of each active agent. In certain embodiments, the first and second therapeutic agents are administered in a single dosage form. In certain embodiments, the first, second, and third therapeutic agents are administered in a single dosage form. The agents may be formulated into a single tablet, pill, capsule, or solution for parenteral administration and the like.

In certain embodiments, the therapeutic agents are administered in a single dosage form, wherein each individual therapeutic agent is isolated from the other therapeutic agent(s). Formulating the dosage forms in such a way assists in maintaining the structural integrity of potentially reactive therapeutic agents until they are administered. A formulation of this type may be useful during production and for long-term storage of the dosage form. In certain embodiments, the therapeutic agents may comprise segregated regions or distinct caplets or the like housed within a capsule. In certain embodiments, the therapeutic agents are provided in isolated layers comprised by a tablet.

Alternatively, the therapeutic agents may be administered as separate compositions, e.g., as separate tablets or solutions. One or more active agent may be administered at the same time as the other active agent(s) or the active agents may be administered intermittently. The length of time between administrations of the therapeutic agents may be adjusted to achieve the desired therapeutic effect. In certain instances, one or more therapeutic agent(s) may be administered only a few minutes (e.g., about 1, 2, 5, 10, 30, or 60 min) after administration of the other therapeutic agent(s). Alternatively, one or more therapeutic agent(s) may be administered several hours (e.g., about 2, 4, 6, 10, 12, 24, or 36 hr) after administration of the other therapeutic agent(s). In certain embodiments, it may be advantageous to administer more than one dosage of one or more therapeutic agent(s) between administrations of the remaining therapeutic agent(s). For example, one therapeutic agent may be administered at 2 hours and then again at 10 hours following administration of the other therapeutic agent(s). Importantly, it is required that the therapeutic effects of each active ingredient overlap for at least a portion of the duration of each therapeutic agent so that the overall therapeutic effect of the combination therapy is attributable in part to the combined or synergistic effects of the combination therapy.

The dosage of the active agents will generally be dependent upon a number of factors including pharmacodynamic characteristics of each agent of the combination, mode and route of administration of active agent(s), the health of the patient being treated, the extent of treatment desired, the nature and kind of concurrent therapy, if any, and the frequency of treatment and the nature of the effect desired. In general, dosage ranges of the active agents often range from about 0.001 to about 250 mg/kg body weight per day. For a normal adult having a body weight of about 70 kg, a dosage in the range of from about 0.1 to about 25 mg/kg body weight is typically preferred. However, some variability in this general dosage range may be required depending upon the age and weight of the subject being treated, the intended route of administration, the particular agent being administered and the like. Since two or more different active agents are being used together in a combination therapy, the potency of each agent and the interactive effects achieved using them together must be considered. Importantly, the determination of dosage ranges and optimal dosages for a particular mammal is also well within the ability of one of ordinary skill in the art having the benefit of the instant disclosure.

In certain embodiments, it may be advantageous for the pharmaceutical combination to have a relatively large amount of the first component compared to the second component. In certain instances, the ratio of the first active agent to second active agent is about 100:1, 90:1, 80:1, 70:1, 60:1, 50:1, 40:1, 30:1, 20:1, 15:1, 10:1, 9:1, 8:1, 7:1, 6:1, or 5:1. In certain embodiments, it may be preferable to have a more equal distribution of pharmaceutical agents. In certain instances, the ratio of the first active agent to the second active agent is about 4:1, 3:1, 2:1, 1:1, 1:2, 1:3, or 1:4. In certain embodiments, it may be advantageous for the pharmaceutical combination to have a relatively large amount of the second component compared to the first component. In certain instances, the ratio of the second active agent to the first active agent is about 30:1, 20:1, 15:1, 10:1, 9:1, 8:1, 7:1, 6:1, or 5:1. In certain instances, the ratio of the second active agent to first active agent is about 100:1, 90:1, 80:1, 70:1, 60:1, 50:1, or 40:1. Importantly, a composition comprising any of the above-identified combinations of first therapeutic agent and second therapeutic agent may be administered in divided doses about 1, 2, 3, 4, 5, 6, or more times per day or in a form that will provide a rate of release effective to attain the desired results. In one embodiment, the dosage form contains both the first and second active agents. In one embodiment, the dosage form only has to be administered one time per day and the dosage form contains both the first and second active agents.

For example, a formulation intended for intravenous administration to humans may contain from about 0.1 mg to about 5 g of the first therapeutic agent and about 0.1 mg to about 5 g of the second therapeutic agent, both of which are compounded with an appropriate and convenient amount of carrier material varying from about 5 to about 95 percent of the total composition. Unit dosages will generally contain between about 0.5 mg to about 1500 mg of the first therapeutic agent and 0.5 mg to about 1500 mg of the second therapeutic agent. In a preferred embodiment, the dosage is about 25 mg, 50 mg, 100 mg, 200 mg, 300 mg, 400 mg, 500 mg, 600 mg, 800 mg, or 1000 mg, etc., up to about 1500 mg of the first therapeutic agent. In a preferred embodiment, the dosage is about 25 mg, 50 mg, 100 mg, 200 mg, 300 mg, 400 mg, 500 mg, 600 mg, 800 mg, or 1000 mg, etc., up to about 1500 mg of the second therapeutic agent.

Dosage amount and interval may be adjusted on an individual or group basis to provide plasma levels of a particular active moiety or moieties sufficient to maintain the modulating effects or minimal effective concentration (MEC) of each of them. The MEC will vary for each compound and individual, but it can be estimated from in vitro data. Dosages necessary to achieve the MEC will depend on individual characteristics and route of administration. However, HPLC assays or bioassays can be used to determine plasma concentrations. In certain embodiments, the dose may be decreased. In certain embodiments, the dose may be increased. Moreover, a long-term treatment regimen may include alternating period of increasing and decreasing dosage with respect to a particular compound or compounds.

### Synergism and Augmentation

The term “synergistic” refers to a combination which is more effective than the additive effects of any two or more single agents. A synergistic effect permits the effective treatment of a disease using lower amounts (doses) of individual therapy. The lower doses result in lower toxicity without reduced efficacy. In addition, a synergistic effect can result in improved efficacy. Finally, synergy may result in an improved avoidance or reduction of disease as compared to any single therapy.

Combination therapy can allow for the product of lower doses of the first therapeutic or the second therapeutic agent (referred to as “apparent one-way synergy” herein), or lower doses of both therapeutic agents (referred to as “two-way synergy” herein) than would normally be required when either drug is used alone.

Combination therapy can allow for the product of lower doses of any one of the therapeutic agents (referred to as “apparent one-way synergy” herein), or lower doses of all therapeutic agents than would normally be required when any drug is used alone.

In certain embodiments, the synergism exhibited between one or more therapeutic agent(s) and the remaining therapeutic agent(s) is such that the dosage of one of the therapeutic agents would be sub-therapeutic if administered without the dosage of the other therapeutic agents.

The terms “augmentation” or “augment” refer to combinations where one of the compounds increases or enhances therapeutic effects of another compound or compounds administered to a patient. In some instances, augmentation can result in improving the efficacy, tolerability, or safety, or any combination thereof, of a particular therapy.

In certain embodiments, the present invention relates to a pharmaceutical composition comprising a therapeutically effective dose of one or more therapeutic agent(s) together with a dose of another therapeutic agent effective to augment the therapeutic effect of the one or more therapeutic agent(s). In other embodiments, the present invention relates to methods of augmenting the therapeutic effect in a patient of one or more therapeutic agent(s) by administering another therapeutic agent to the patient.

In certain preferred embodiments, the invention is directed in part to synergistic combinations of one or more therapeutic agent(s) in an amount sufficient to render a therapeutic effect together with the remaining therapeutic agent(s). For example, in certain embodiments a therapeutic effect is attained which is at least about 2 (or at least about 4, 6, 8, or 10) times greater than that obtained with the dose of the one or more therapeutic agent(s) alone. In certain embodiments, the synergistic combination provides a therapeutic effect which is up to about 20, 30 or 40 times greater than that obtained with the dose of the one or more therapeutic agent(s) alone. In such embodiments, the synergistic combinations display what is referred to herein as an “apparent one-way synergy”, meaning that the dose of the remaining therapeutic agent(s) synergistically potentiates the effect of the one or more therapeutic agent(s), but the dose of the one or more therapeutic agent(s) does not appear to significantly potentiate the effect of the remaining therapeutic agent(s).

In certain embodiments, the combination of active agents exhibits two-way synergism, meaning that the second therapeutic agent potentiates the effect of the first therapeutic agent, and the first therapeutic agent potentiates the effect of the second therapeutic agent. Thus, other embodiments of the invention relate to combinations of a second therapeutic agent and a first therapeutic agent where the dose of each drug is reduced due to the synergism between the drugs, and the therapeutic effect derived from the combination of drugs in reduced doses is enhanced. The two-way synergism is not always readily apparent in actual dosages due to the potency ratio of the first therapeutic agent to the second therapeutic agent. For instance, two-way synergism can be difficult to detect when one therapeutic agent displays much greater therapeutic potency relative to the other therapeutic agent.

The synergistic effects of combination therapy may be evaluated by biological activity assays. For example, the therapeutic agents are mixed at molar ratios designed to give approximately equipotent therapeutic effects based on the EC₉₀ values. Then, three different molar ratios are used for each combination to allow for variability in the estimates of relative potency. These molar ratios are maintained throughout the dilution series. The corresponding monotherapies are also evaluated in parallel to the combination treatments using the standard primary assay format. A comparison of the therapeutic effect of the combination treatment to the therapeutic effect of the monotherapy gives a measure of the synergistic effect. Further details on the design of combination analyses can be found in B E Korba (1996) Antiviral Res. 29:49. Analysis of synergism, additivity, or antagonism can be determined by analysis of the aforementioned data using the CalcuSyn™ program (Biosoft, Inc.). This program evaluates drug interactions by use of the widely accepted method of Chou and Talalay combined with a statistically evaluation using the Monte Carlo statistical package. The data are displayed in several different formats including median-effect and dose-effects plots, isobolograms, and combination index [CI] plots with standard deviations. For the latter analysis, a CI greater than 1.0 indicates antagonism and a CI less than 1.0 indicates synergism.

Compositions of the invention present the opportunity for obtaining relief from moderate to severe cases of disease. Due to the synergistic or additive or augmented effects provided by the inventive combination of the first and second therapeutic agent, it may be possible to use reduced dosages of each of therapeutic agent. Due to the synergistic or additive or augmented effects provided by the inventive combination of the first, second, and third therapeutic agents, it may be possible to use reduced dosages of each of therapeutic agent. By using lesser amounts of drugs, the side effects associated with each may be reduced in number and degree. Moreover, the inventive combinations avoid side effects to which some patients are particularly sensitive.

### Pharmaceutical Compositions and Formulations

In certain embodiments, the invention also provides pharmaceutical compositions comprising one or more compounds described herein in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. It is also envisioned that the compounds may be incorporated into transdermal patches designed to deliver the appropriate amount of the drug in a continuous fashion.

For preparing solid compositions such as powders and tablets, the principal active ingredient is mixed with a pharmaceutically acceptable carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture. When referring to these preformulation compositions as homogeneous, it is meant that the active ingredient is dispersed evenly throughout the composition so that the composition may be easily subdivided into equally effective unit dosage forms.

In some embodiments, a dry powder composition is micronized for inhalation to the lungs. See for example, U.S. Patent Application publication 2016/0263257, expressly incorporated herein by reference in its entirety, and in particular regarding the dry powder cromolyn formulations described therein. In other embodiments, the dry powder composition further comprises at least one excipient. In certain embodiments, the at least one excipient comprises Lactose monohydrate and/or Magnesium stearate.

The phrase “therapeutically-effective amount” as used herein means that amount of a therapeutic agent in a composition of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or stearic acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the compounds found in the present compositions may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds comprised in compositions of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the compounds that the present compositions comprise include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isothionic, and the like.

In other cases, the compounds comprised in compositions of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredients which can be combined with a carrier material to produce a single dosage form will vary depending upon the host being treated, the particular mode of administration. The amount of active ingredients which can be combined with a carrier material to produce a single dosage form will generally be those amounts of the compounds which produce a therapeutic effect. Generally, out of one hundred percent, this amount will range from about 0.1 percent to about ninety-nine percent of active ingredients, preferably from about 5 percent to about 70 percent, most preferably from about 10 percent to about 30 percent.

Methods of preparing these formulations or compositions include the step of bringing into association two or more active compounds with the carrier and, optionally, one or more accessory ingredients. In general, the formulations are prepared by uniformly and intimately bringing into association one or more active compounds with liquid carriers, or finely divided solid carriers, or both, and then, if necessary, shaping the product.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise two or more therapeutic agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the product of coating materials, such as lecithin, by the maintenance of the required particle size in the case of dispersions, and by The product of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The compositions comprising the two or more therapeutic agents can be, alone or in combination with other therapeutic agents, employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., other analgesic agents. For parenteral application, particularly suitable are oily or aqueous solutions, as well as suspensions, emulsions, or implants, including suppositories. Ampoules are convenient unit dosages. For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay release of the active ingredients. Formulations for oral use may also be presented as hard gelatin capsules wherein the active ingredients are mixed with an inert diluent.

Aqueous suspensions contain the above-identified combinations of drugs and that mixture has one or more excipients suitable as suspending agents, for example pharmaceutically acceptable synthetic gums such as hydroxypropylmethylcellulose or natural gums. Oily suspensions may be formulated by suspending the above-identified combination of drugs in a vegetable oil or mineral oil. The oily suspensions may contain a thickening agent such as beeswax or cetyl alcohol. A syrup, elixir, or the like can be used wherein a sweetened vehicle is employed. Injectable suspensions may also be prepared, in which case appropriate liquid carriers, suspending agents and the like may be employed. It is also possible to freeze-dry the active compounds and use the obtained lyophilized compounds, for example, for the preparation of products for injection.

One aspect of combination therapy pertains to a method for providing effective therapeutic treatment in humans, comprising administering an effective or sub-therapeutic amount of one or more therapeutic agent(s); and administering the remaining therapeutic agent(s) in an amount effective to augment the therapeutic effect provided by said one or more therapeutic agent(s). The therapeutic agents can be administered simultaneously or at different times, as long as the dosing intervals (or the therapeutic effects) of the therapeutic agents overlaps. In other words, according to the method of the present invention, in certain preferred embodiments the therapeutic agents need not be administered in the same dosage form or even by the same route of administration as each other. Rather, the method is directed to the surprising synergistic and/or additive benefits obtained in humans, when therapeutically effective levels of one or more therapeutic agent(s) have been administered to a human, and, prior to or during the dosage interval for the therapeutic agent(s) or while the human is experiencing the therapeutic effect, an effective amount of other therapeutic agent(s) to augment the therapeutic effect of the original one or more therapeutic agent(s) is administered.

Injectable depot forms are made by forming microencapsule matrices of the subject compounds in biodegradable polymers such as polylactide-polyglycolide. Depending on the ratio of drugs to polymer, and the nature of the particular polymer employed, the rate of drug release can be controlled. Examples of other biodegradable polymers include poly(orthoesters) and poly(anhydrides). Depot injectable formulations are also prepared by entrapping the drugs in liposomes or microemulsions which are compatible with body tissue.

The preparations of the present invention may be given orally, parenterally, topically, or rectally. They are of course given in forms suitable for each administration route.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Subcutaneous administration is preferred.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Actual dosage levels of the active ingredients in the pharmaceutical compositions of this invention may be varied so as to obtain an amount of an active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the active compounds employed in the pharmaceutical composition at levels lower than that required in order to achieve the desired therapeutic effect and gradually increase the dosage until the desired effect is achieved.

While it is possible for an active compound of the present invention to be administered alone, it is preferable to administer the compound as a pharmaceutical formulation (composition).

The patient receiving this treatment is any animal in need, including primates, in particular humans, and other mammals such as equines, cattle, swine and sheep; and poultry and pets in general.

In certain embodiments, the present invention relates to a method of treating a disease or condition in a subject in need thereof comprising co-administering a therapeutically effective amount of a pharmaceutical composition comprising the first compound and a therapeutically effective amount of a pharmaceutical composition comprising of the second compound. In certain embodiments the disease or condition is a neuron inflammation condition.

In certain embodiments, the present invention relates to a method of slowing the progression of a disease or condition in a subject in need thereof comprising co-administering a therapeutically effective amount of a first pharmaceutical composition comprising the first compound and a therapeutically effective amount of a second pharmaceutical composition comprising of the second compound. In certain embodiments the disease or condition is a neuron inflammation condition.

In certain embodiments, the first compound has the following formula (I):

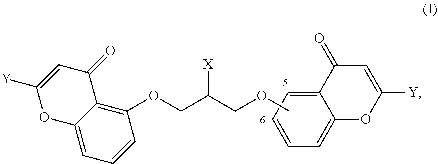

wherein

X is halide, hydroxyl, or OCO(C₁₋₈alkyl);

Y is CO₂R¹ or CH₂OR²;

R¹ is Li, Na, K, H, C₁₋₃alkyl, —CH₂CO₂(C₁₋₅alkyl); and

R² is H or —C(O)(C₁₋₃alkyl).

In certain embodiments the present invention relates to the pharmaceutically acceptable salts of compound of formula (I).

In certain embodiments the compound of formula (I) is selected from:

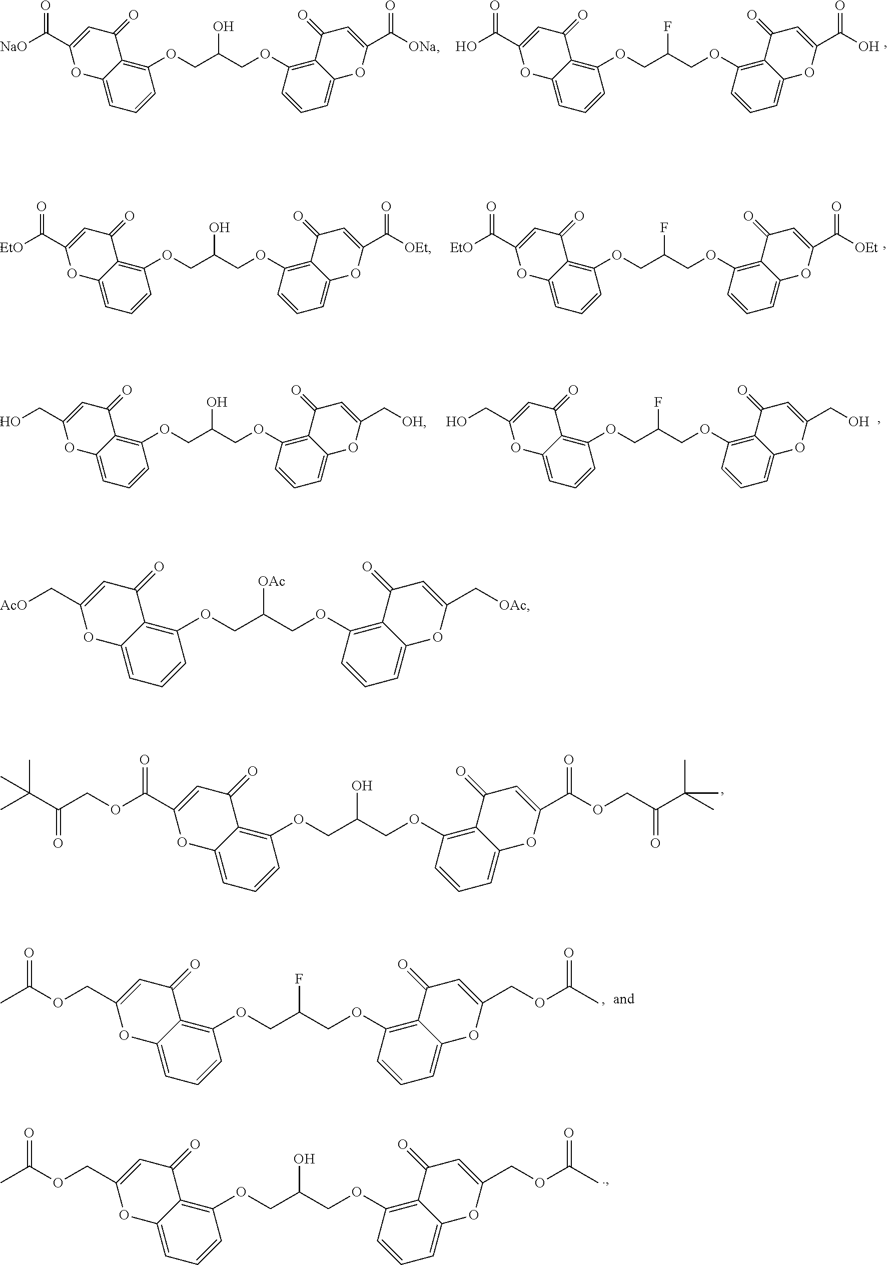

or a pharmaceutically acceptable salt thereof.

In certain embodiments, the first compound is selected from: bitolterol, fenoterol, isoprenaline, levosalbutamol, orciprenaline, pirbuterol, procaterol, ritodrine, salbutamol, terbutaline, arformoterol, bambuterol, clenbuterol, formoterol, salmeterol, abediterol, carmoterol, indacaterol, olodaterol, vilanterol, nedocromil, ketotifen, olopatadine, omalizumab, quercetine, mepolizumab, azelastine, methylxanthines, pemirolast, olopataidne, alfatoxin G₁, alfatoxin B₁, alfatoxin M₁, deoxynivalenol, zearalenone, ochratoxin A, fumonisin B₁, hydrolyzed fumonisin B₁, patulin, and ergotamine.

In certain embodiments, the first compound is selected from the following compounds or pharmaceutically acceptable salts thereof:

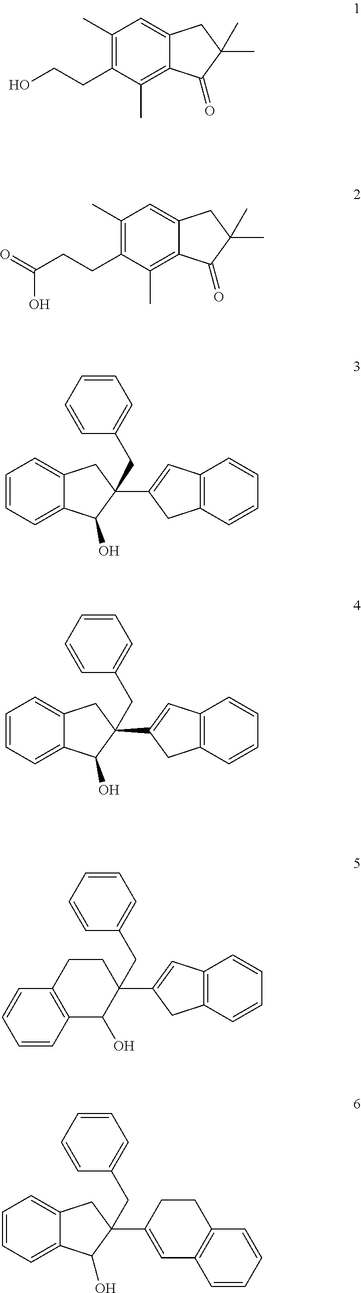 [SMILES: Cc1cc2c(c(C)c1CCO)C(=O)C(C)(C)C2 ; Cc1cc2c(c(C)c1CCC(=O)O)C(=O)C(C)(C)C2 ; O[C@H]1c2ccccc2C[C@]1(Cc1ccccc1)C1=Cc2ccccc2C1 ; O[C@H]1c2ccccc2C[C@@]1(Cc1ccccc1)C1=Cc2ccccc2C1 ; OC1c2ccccc2CCC1(Cc1ccccc1)C1=Cc2ccccc2C1 ; OC1c2ccccc2CC1(Cc1ccccc1)C1=Cc2ccccc2CC1]

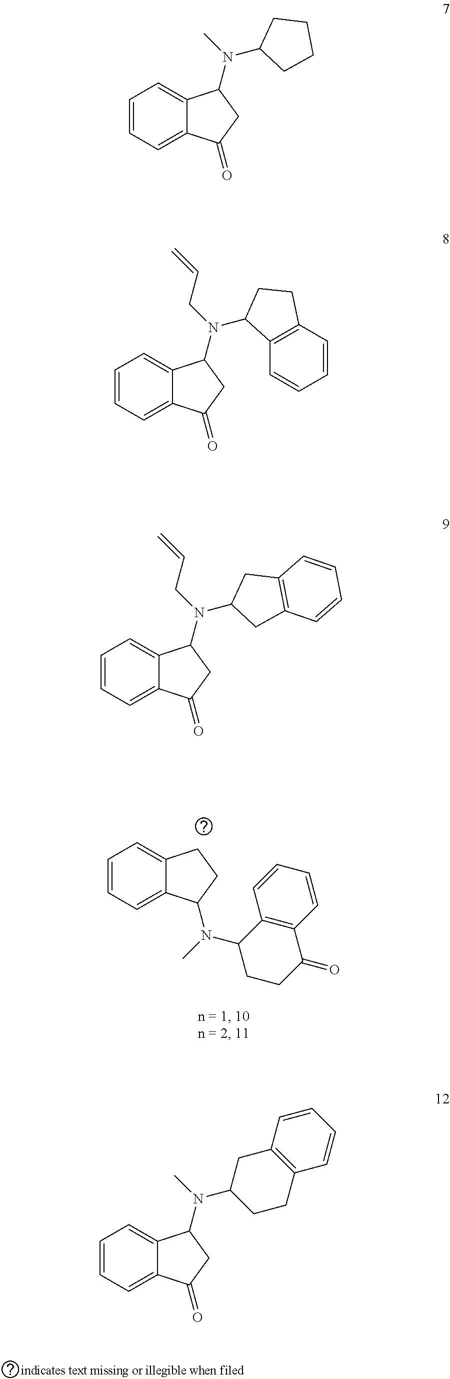 [SMILES: CN(C1CCCC1)C1CC(=O)c2ccccc21 ; C=CCN(C1CCc2ccccc21)C1CC(=O)c2ccccc21 ; C=CCN(C1Cc2ccccc2C1)C1CC(=O)c2ccccc21 ; CN(C1CCc2ccccc21)C1CCC(=O)c2ccccc21 ; CN(C1CCc2ccccc2C1)C1CC(=O)c2ccccc21]

In certain embodiments, the first compound is selected from edaravone and riluzole.

In certain embodiments, the second compound is selected from non-steroidal anti-inflammatory drugs (NSAID).

In certain embodiments, the second compound is selected from: acetylsalicylic acid, diflunisal, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, celecoxib, licofelone, hyperforin, and figwort.

In certain embodiments, the second compound is selected from anti-inflammatory small molecular peptides truncated from an anti-inflammatory gene protein, such as TREM2.

In certain embodiments, the present invention relates to a method comprising co-administering a plurality of pharmaceutical compositions of the compounds designated as “first compound” and, optionally, a plurality of pharmaceutical compositions of the compounds designated as “second compound”. For example, a composition comprising compound of formula (I) can be co-administered with a composition comprising edaravone. Alternatively, a composition comprising compound of formula (I) can be co-administered with a composition comprising riluzole. In certain embodiments, a composition comprising compound of formula (I) can be co-administered with a composition comprising riluzole and a composition comprising an NSAID, such as ibuprofen or meloxicam. In some embodiments, a composition comprising compound of formula (I) can be co-administered with a composition comprising edaravone and a composition comprising an NSAID, such as ibuprofen or meloxicam.

In certain embodiments, the neuron inflammation condition is ALS, autism spectrum disorder (ASD), ischemic stroke, or prion disease. For example, the neuron inflammation condition is ALS. Alternatively, the neuron inflammation condition is a prion disease.

In certain embodiments, the claimed methods result in slowing down neuron damage for neurons located in the brain stem and/or the spinal cord, neurons, or motor neurons that affect voluntary body muscles.

In certain embodiments, the claimed methods result in halting the damage for neurons located in the brain stem and/or the spinal cord, neurons, or motor neurons that affect voluntary body muscles.

In certain embodiments, the compounds or compositions are administered subcutaneously, intravenously, intraperitoneally, by inhalation, orally, or transdermally. For example, the composition is administered subcutaneously. Alternatively, the composition is administered intravenously.

In certain embodiments, the compounds or compositions are administered in doses specifically tailored to lead to blood, brain, and CSF concentrations that allow the drugs to act as M1-to-M2 modifiers.

In certain embodiments, the claimed methods result in improvement of body function or reduction of the symptoms associated with brain regions that control motor neurons and affect ALS manifestation. In certain embodiments, the claimed methods result in improvement of the mood and social behavior in patients suffering from ALS.

### Examples

### Methods

### Chemicals

Cromolyn sodium was provide by AZTherapies and dissolved in PBS. 100 mM solution was used for in vivo experiments. Dulbecco's PBS was used to dilute the solution for intraperitoneal injections for a final dose of 6.3 mg/kg as described previously.

### Animals

149 mice were used in this study. All animal care, husbandry and experimentation were performed according to the guidelines set by the Massachusetts General Hospital Subcommittee on Research Animal Care. These experiments were approved by the Massachusetts General Hospital Institutional Animal Care and Use Committee (2014N000018). All mice were given access to food and water ad libitum. Mice were assessed regularly for motor impairment and euthanized upon onset of major paralysis (neurological scoring=4, see Neurological Score below) to minimize suffering as described previously. Mice that exhibited mild paralysis (neurological score=2) were given water bottles with long sipper tubes and hydrogel. The endpoint used in this study was based on previously reported criteria by ALS TDI, the loss of self-righting ability within 15 seconds (neurological score=4) or the inability to move to reach food on the cage floor. Mice that reached the humane endpoint were euthanized within 3 hours.

SOD1^{G93A} mice:

B6SJL-Tg (SOD1 G93A)1Gur/J transgenic male mice were obtained from Jackson Laboratory and bred with C57BL/6 female mice to obtain wild-type (Wt) SOD1 and mutant transgenic (Tg) SOD1^{G93A}-expressing mice. To determine mouse genotype, RNA extraction and complimentary DNA (cDNA) synthesis was performed from tail biopsies acquired at postnatal day 28-40 followed by quantitative real-time PCR (qRT-PCR) using primers for the mutant G93A SOD1 gene (GGGAAGCTGTTGTCCCAAG and CAAGGGGAGGTAAAAGAGAGC). Both age- and litter-matched WtSOD1 and TgSOD1 male and female mice were used for all studies as described below.

### Behavior

All behavioral assessments, data collection, and analysis were carried out by an investigator who was blind to the experimental conditions (i.e., genotypes and treatment).

Gait Analysis Manual gait analysis was performed using a limb painting procedure similar to previous studies. Briefly, mice were first trained to traverse a horizontal corridor leading directly into their home cage by gentle nudges in the appropriate direction. On test days the bottoms of their hindlimbs were painted, by brushing with non-toxic food dye (Fisher Scientific), and the mice were allowed to walk the path to their home cage on a piece of paper. Three trials were performed at each experimental time point (P70, P90, P110, P130, P150). Stride length and width was determined by measuring the distance between the same points, on the ball mount region of the footprint, in two consecutive footprints and calculated from 2-3 hindpaw strides. Mean data from 4-6 strides across three trials was calculated.

### Rotarod

Mice were placed on a fixed speed (16 rpm) rotating rod (3.0 cm) (Rotamex, Columbus Instruments) as previously described. Mice were trained to remain on the rotarod for 180 seconds once at P40. For each experimental time point (P70, P90, P110, P130, P150), the time mice spent on the rotating rod was calculated up to a maximum of 180 seconds. Three trials were performed for each time point and the greatest value for each session was used for analysis.

### Paw Grip Endurance Test (PaGE)

The PaGE test was performed as previously described. Briefly, mice were placed on the wire lid of a conventional housing cage that was inverted and held at ˜45 cm above an open cage bottom. For experimental time points (P70, P90, P110, P130, P150), the time spent on the grid (before falling) was noted up to a maximum value of 90 seconds. The largest value from three individual trials was used for analysis.

### Weight and Neurological Scoring

Beginning at P50, weight and neurological score (using the ALS TDI criteria) (Hatzipetros, T., et al. (2015). “A Quick Phenotypic Neurological Scoring System for Evaluating Disease Progression in the SOD1-G93A Mouse Model of ALS.” J Vis Exp(104); Leitner M., et. al. (2009). “Working with ALS mice: Guidelines for preclinical testing & colony management.” The Jackson Laboratory) were recorded for each mouse every 5 days until death or euthanasia. ALS TDI criteria are as follows:
Score of 0: Full extension of hind legs away from lateral midline when mouse is suspended by its tail, and mouse can hold this for two seconds, suspended two to three times.
Score of 1: Collapse or partial collapse of leg extension towards lateral midline (weakness) or trembling of hind legs during tail suspension.
Score of 2: Toes curl under at least twice during walking of 12 inches, or any part of foot is dragging along cage bottom/table.
Score of 3: Rigid paralysis or minimal joint movement, foot not being used for generating forward motion.
Score of 4: Mouse cannot right itself within 15 seconds after being placed on either side.
   *Mice were euthanized upon obtaining a score of 4.

### Tissue Dissection

Tissue was dissected from TgSOD1^{G93A} mice upon reaching a neurological score of 4). Mice were sacrificed by administration of slow flow CO₂ (10-30% of the chamber volume/minute) followed by immediate decapitation. Brain, gastrocnemius, and tibialis anterior tissue were removed and frozen in dry ice. Spinal cord was removed, frozen by gently lowering into the gas byproduct of liquid nitrogen, and dissected into lumbar and non-lumbar regions. All tissue was stored at −80° C. prior to use. In addition, tail samples were extracted to perform a second round of confirmatory qRT-PCR for mouse genotyping. Tail samples were stored at −20° C. until used.

### Motor Neuron Quantification

Longitudinal sections (10 μm) of the lumbar spinal cord were cut from fresh frozen tissue. Hematoxylin and eosin (H&E) staining was performed on the tissue sections as previously described. Within the region containing the ventral horn, all motor neurons are counted for three individual sections per animal (each separated by 20-30 m) to cover the areas of highest motor neuron density within the ventral horn. All counting was performed by an individual blinded to the genotypes. Images were acquired using a Zeiss microscope 20× objective (0.8 NA) and processed with Metamorph image analysis software (Molecular Devices).

### Iba1 Immunohistochemical 516 Analysis

Frozen spinal cord sections were fixed in 4% PFA/PBS for 72 hours and dehydrated with 30% sucrose in PBS. Sections were washed three times (5 minutes each) with PBS and incubated with 3% H₂O₂ in PBS for 15 minutes to quench endogenous peroxidases. Sections were subsequently washed three times with PBS and blocked using 5% (v/v) normal goat serum (Vector Laboratories), 0.3% Triton X-100 in PBS. Primary antibody against Iba1 (rabbit polyclonal, 1:400, Wako, #019-19741) was diluted in a buffer containing 2.5% (v/v) normal goat serum, 0.3% Triton X-100 and incubated overnight at 4° C. On the following day, samples were washed three times (10 minutes each) with PBS. The primary antibody was detected using a biotinylated secondary antibody (1:200) and VECTASTAIN Elite ABC HRP kit (Vector Laboratories), and developed with DAB (Vector Laboratories), following the provider's instructions. Sections were dehydrated in a series of graded ethanol, cleared in xylene, and cover-slipped with Cytoseal-XYL xylene-based mounting medium (Thermo Fisher Scientific). Sections were imaged using a light microscope (TE360 Eclipse; Nikon, Japan) at 10× magnification. The Iba1-positive cell area (area occupied by Iba1-positive cells divided by the total area) was quantified for each spinal cord section using ImageJ software (Voxel counter plugin, NIH, USA). Two to three sections were analyzed per mouse. Values from each section were averaged to obtain a mean value for each animal.

### Meso Scale Discovery Multi-Spot Cytokine Assay

Spinal cord frozen tissue was homogenized in ice-cold RIPA buffer (Thermo Fisher Scientific, #8990) supplemented with protease inhibitor cocktail (Thermo Fisher Scientific, #78430). Samples were centrifuged at 45,000 g for 30 minutes at 4° C. using an Optima TL ultracentrifuge and a TLA 120.2 rotor (Beckman Coulter). Expression levels of 10 pro-inflammatory cytokines and chemokines were assessed in the supernatants derived from spinal cord tissue or in the plasma, using an electrochemiluminescence-based multi-array method and MESO Quickplex SQ 120 system (MSD, Rockville, Md., USA). The 96-well V-PLEX Proinflammatory Mouse 1 Kit (Meso Scale Discovery, #K15048D) was used to measure simultaneously IFN-γ, IL-1β, IL-2, IL-4, IL-5, IL-6, CXCL1/KC/GRO, IL-10, IL-12, p70, and TNFα, following the manufacturer's instructions. Briefly, samples were diluted in the calibrator and added to the plate coated with an array of cytokine capture antibodies. Samples were incubated in the plate for 2 hours with shaking at room temperature, followed by washes with the wash buffer provided in the kit. The detection antibody solution was added to each well and the plate was incubated for 2 hours. The plate was washed with the wash buffer and the 2× Read Buffer T was added. The signal was immediately measured on a MESO QuickPlex SQ 120 instrument and was analyzed using the DISCOVERY WORKBENCH 4.0 software (Meso Scale Diagnostics, LLC., Rockville, Md., USA). Protein concentrations in the supernatants or the plasma samples were measured using the Pierce BCA protein assay kit (Thermo Scientific). Values in the graphs represent levels of cytokines normalized to the corresponding protein concentrations.

### CCL2/MCP-1 ELISA Assay

Spinal cord tissue was homogenized, and supernatants were derived as described in the section related to the Meso Scale Discovery cytokine assay. MCP-1 levels were measured in the supernatants generated from spinal cord tissue or in the plasma using the 96-well Mouse CCL2/JE/MCP-1 Quantikine ELISA Kit (R&D systems, #MJE00B), following the manufacturer's instructions. Briefly, samples and diluted standards were added to the plate coated with MCP-1-specific antibody. Samples were incubated in the plate for 2 hours on a shaker at room temperature, followed by washes with the wash buffer provided in the kit. Subsequently, horseradish peroxidase conjugated antibody against MCP-1 was added to each well and incubated for 2 hours at room temperature on the shaker. The plate was washed with the wash buffer and incubated with Substrate solution for 30 minutes at room temperature. Next, Stop solution was added and the signal was read on a Microplate reader (Synergy 2, Biotek Instruments). The optical density was measured at 405 nm and was corrected with the optical density measured at 540 nm. MCP-1 levels in the samples were calculated based on the MCP-1 standard curve. Protein concentrations in the supernatants were measured by the Pierce BCA protein assay kit (Thermo Scientific). Values in the graphs represent levels of MCP-1 normalized to the corresponding protein concentrations.

### GPR35 Western Blots

Western blots were performed as previously described (Mueller et al., 2018). Briefly, 75 g of mouse spinal cord protein was re-suspended in sample buffer, boiled at 95° C. for 5 minutes, and fractioned on 10-20% Tricine gels (Life Technologies), and the membrane was then blocked with 5% milk in tris-buffered saline with Tween 20 (TBST) before immunodetection with antibodies specific for the following: GPR35 (Novus Biologicals, Littleton, Colo.; NBP2-24640), and beta-actin (Cell Signaling Technology, Danvers, Mass.; 4967S). Primary antibody incubation overnight was followed by 4 washes (15 min, RT) in TBST before incubation with the secondary antibody for 1 h (HRP-conjugated goat anti-rabbit IgG Jackson ImmunoResearch Laboratories, West Grove, Pa.; and HRP-conjugated goat anti-mouse Bio-Rad Laboratories Hercules, Calif.). After 4 washes in TBST (15 min, RT) proteins were visualized using the ECL detection system (NEN, Boston, Mass.). GPR35 integrated density values (IDV) were normalized to beta-actin.

### GPR35 Immunofluorescence

Frozen mouse lumbar spinal cord was sectioned at 10 m and stained using previously described methods. Tissue sections were fixed with 4% paraformaldehyde and blocked in a mixture of phosphate-buffered saline (PBS), 5% BSA, and normal goat serum. Co-staining was done using antibodies specific for GPR35 (Novus Biologicals, Littleton, Colo.; NBP2-24640) and NeuN (Millipore Sigma, Temecula, Calif.; MAB377 overnight at 4° C. Following three washes with PBS, sections were incubated with Cy3-conjugated goat anti-mouse and FITC-conjugated goat anti-rabbit antibodies (Jackson ImmunoResearch, West Grove, Pa.). Ten 20× fields were imaged from each section and analyzed using open source software from the National Institutes of Health (ImageJ).

### Statistics

Data in the main text are presented as median values. Box plots are used for graphical representation of population data with the central line representing the median, the edges representing the interquartile ranges, and the whiskers representing 10-90th percentiles. Data are also represented as medians interquartile ranges or percent values. Sample sizes are included in the figure legends. Comparisons for unrelated samples were performed using a two-way ANOVA followed by Tukey's or Sidak's multiple comparison's test or a one-way ANOVA test followed by Tukey's multiple comparison post-tests at a significance level (a) of 0.05. For p<0.05 and >0.00001, exact P values (two-tailed) are reported.

### Study Approval

All animal studies were approved by the Massachusetts General Hospital Institutional Animal Care and Use Committee (2014N000018).

### Results

149 male and female age- and litter-matched transgenic (Tg) SOD1^{G93A} and wild-type (Wt) SOD1^{G93A} mice were used with the following breakdown: Females (19 WtSOD1-Vehicle, 17 WtSOD1-Cromolyn, 19 TgSOD1-Vehicle, and 17 TgSOD1-Cromolyn) and Males (18 WtSOD1-Vehicle, 21 WtSOD1-Cromolyn, 21 TgSOD1-Vehicle, 17 TgSOD1-Cromolyn). The mice received once daily injections of either vehicle or cromolyn sodium (6.3 mg/kg, 96 i.p.) 5 days per week starting at P60 until euthanasia.

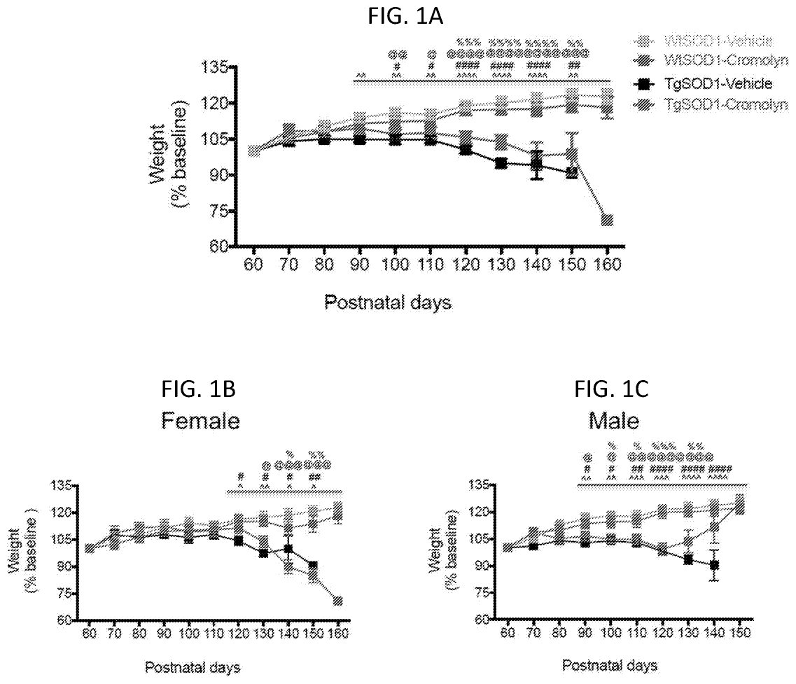

Cromolyn sodium treatment does not alter body weight of TgSOD1 mice First, the effect of cromolyn sodium treatment on body weight was assessed in each group. Two-way ANOVA demonstrated a significant effect of age [F(9, 1143)=10.58, p<0.0001], treatment [F(3, 1143)=47.99, p<0.0001], and age X treatment interaction effect [F(27, 1143)=4.578, p<0.0001] on body weight. Tukey's multiple comparison test revealed that there was a significant decrease in body weight in the TgSOD1-Vehicle group compared to both WtSOD1-Vehicle and WtSOD1-Cromolyn at P100, P110, P120, P130, P140, and P150 (FIG. 1A-FIG. 1C). There was also a significant decrease in body weight in the TgSOD1-Cromolyn group compared to WtSOD1-Cromolyn group at P100, P110, P120, P130, P140, and P150. There was a significant difference in body weight between the TgSOD1-Cromolyn group and WtSOD1-Vehicle group at P120, P130, and P140 only. Importantly, there was a significant improvement in body weight in the TgSOD1-Cromolyn group compared to TgSOD1-Vehicle group at P130, suggesting that cromolyn treatment delayed body weight loss in the treated mice at this timepoint (FIG. 1A). We also assessed the effect of treatment on body weight in female and male mice separately. Two-way ANOVA demonstrated a significant effect of age [F(9, 524)=5.686, p<0.0001], treatment [F(3, 524)=13.76, p<0.0001], and age X treatment interaction effect [F(27, 524)=4.578, p<0.0001] on body weight in female mice. Tukey's multiple comparison test revealed that there was a significant decrease in body weight in the TgSOD1-Vehicle group compared to wild-type groups at P120 and P130 (FIG. 1). There was also a significant decrease in body weight in the TgSOD1-Cromolyn group compared to wild-type groups at P130, P140, and P150. Two-way ANOVA in male mice revealed a significant effect of age [F(8, 549)=7.11, p<0.0001], treatment [F(3, 549)=58.48, p<0.0001], and age X treatment interaction effect [F(24, 549)=3.623, p<0.0001] on body weight in male mice. Tukey's multiple comparison test revealed that there was a significant decrease in body weight in the TgSOD1-Vehicle group compared to wild-type groups at P90, P100, P110, P120, P130, and P140 (FIG. 1C). There was also a significant decrease in body weight in the TgSOD1-Cromolyn group compared to wild-type groups at P90, P100, P110, P120, and P130 (FIG. 1C). Thus, cromolyn treatment did not impact body weight of TgSOD1 mice, except for at P130 when a significant improvement was observed.

### Cromolyn Sodium Treatment Improved Neurological Score and Delayed Disease Onset in TgSOD1 Mice

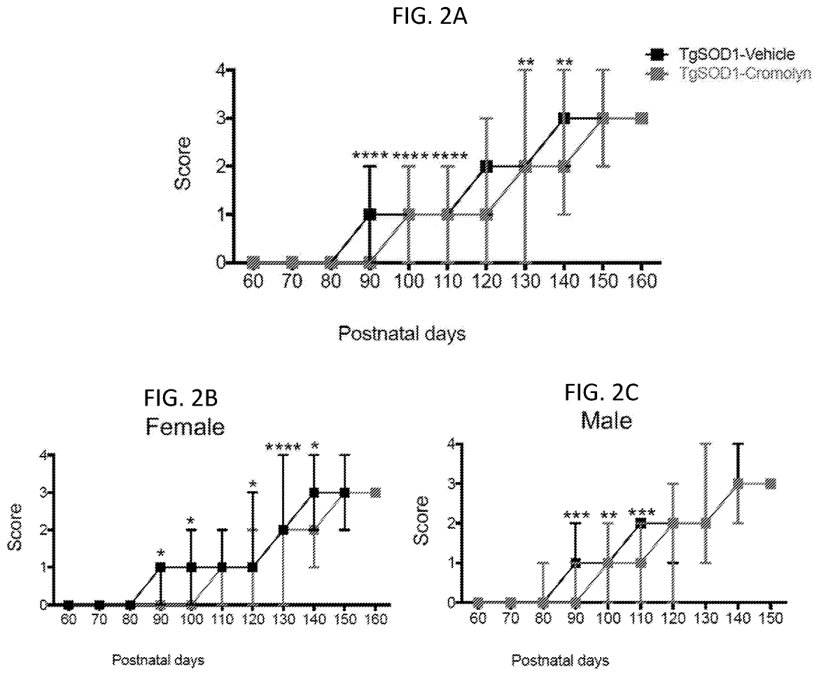

Next, we assessed alterations in neurological score following cromolyn sodium treatment. Two-way ANOVA demonstrated a significant effect of age [F(9, 548)=172.3,p<0.0001], treatment [F(1, 548)=35.32, p<0.0001], and a significant age X treatment interaction on neurological score [F(9, 548)=4.739, p<0.0001]. Tukey's post-hoc analysis revealed a significant increase in neurological score in the TgSOD1-Vehicle treated group compared to TgSOD1-Cromolyn group at P90, P100, P110, P130, and P140, suggesting that cromolyn treatment significantly delayed disease onset and progression (FIG. 2A). Two-way ANOVA demonstrated a significant effect of age [F(9, 269)=91.83, p<0.0001], treatment [F(1, 269)=31.99, p<0.0001], and a significant age X treatment interaction [F(9, 269)=3.175, p<0.0012] on neurological score in female mice. Tukey's post-hoc analysis revealed a significant increase in neurological score in the female TgSOD1-Vehicle treated group compared to TgSOD1-Cromolyn group at P90, P100, P120, P130, and P140 (FIG. 2B). Similar to female mice, two-way ANOVA demonstrated a significant effect of age [F(8, 260)=96.81, p<0.0001], treatment [F(1, 260)=15.99, p<0.0001], and a significant age X treatment interaction [F(8, 260)=3.801, p=0.0003] on neurological score in male mice. Tukey's post-hoc analysis revealed a significant increase in neurological score in male TgSOD1-Vehicle treated group compared to TgSOD1-Cromolyn group at P90, P100, and P110 (FIG. 2C). These findings suggest that cromolyn treatment delayed disease onset and progression in TgSOD1 mice.

### Cromolyn Sodium Treatment Improved Performance on PAGE Task but Did not Alter Rotarod or Gait Performance

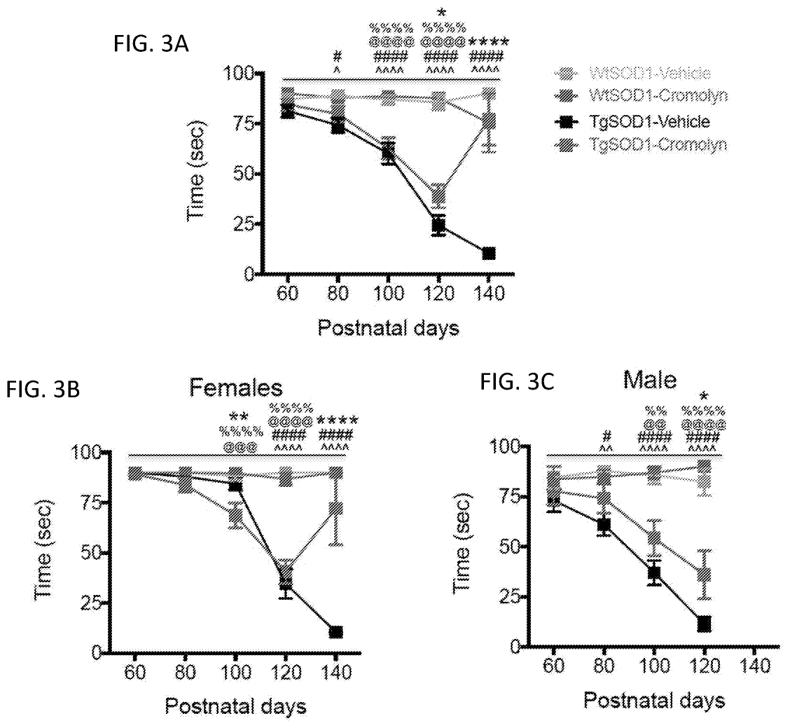

The effect of cromolyn sodium treatment was also assessed on alterations in muscle strength using the paw grip endurance (PaGE) task. Two-way ANOVA demonstrated a significant effect of age [F(4, 492)=31.73, p<0.0001], treatment [F(3, 492)=48.49, p<0.0001] and a significant age X treatment interaction on PaGE [F(12, 492)=10.89, p<0.0001] (FIG. 3A). Tukey's post-hoc analysis revealed a significant decrease in PaGE in the TgSOD1-Vehicle group at P80, P100, P120, and P140 compared to WtSOD1-Vehicle and WtSOD1-Cromolyn groups (FIG. 3A). In addition, there was a significant decrease in PaGE performance in the TgSOD1-Cromolyn group compared to both wild-type groups at P100 and P120. Importantly, there was a significant improvement in PaGE performance in TgSOD1-Cromolyn compared to TgSOD1-Vehicle group at P120 and P140 (FIG. 3A). Similarly, two-way ANOVA demonstrated a significant effect of age [F(4, 274)=41.14, p<0.0001], treatment [F(3, 274)=53.41, p<0.0001] and a significant age X treatment interaction on PaGE performance in female mice [F(12, 274)=16.2, p<0.0001] (FIG. 3B). Tukey's post-hoc analysis revealed a significant decrease in PaGE performance in the TgSOD1-Vehicle group at P120 and P140 compared to WtSOD1-Vehicle and WtSOD1-Cromolyn groups (FIG. 3B). In addition, there was a significant decrease in PaGE performance in the TgSOD1-Cromolyn group compared to both wild-type groups at P100 and P120. Importantly, there was a statistically significant difference between TgSOD1-Vehicle and TgSOD1-Cromolyn at P100 where the cromolyn treated group demonstrated greater deficits in PaGE performance, and a significant improvement at P140 in the treated group (FIG. 3B). In male mice, two-way ANOVA demonstrated a significant effect of age [F(3, 208)=13.34, p<0.0001], treatment [F(3, 208)=48, p<0.0001] and a significant age X treatment interaction on PaGE [F(9, 208)=5.828, p<0.0001] (FIG. 3C). Tukey's post-hoc analysis revealed a significant decrease in PaGE in the TgSOD1-Vehicle group compared to both wild-type groups at P80, P100, and P120. There was also a significant decrease in PaGE in the TgSOD1-Cromolyn group compared to both wild-type groups at P100 and P120. Importantly, there was a significant improvement in PaGE at P120 between the two male transgenic groups (FIG. 3C). Thus, cromolyn treatment improved PaGE performance in treated TgSOD1 mice as compared to the vehicle treated TgSOD1 group.

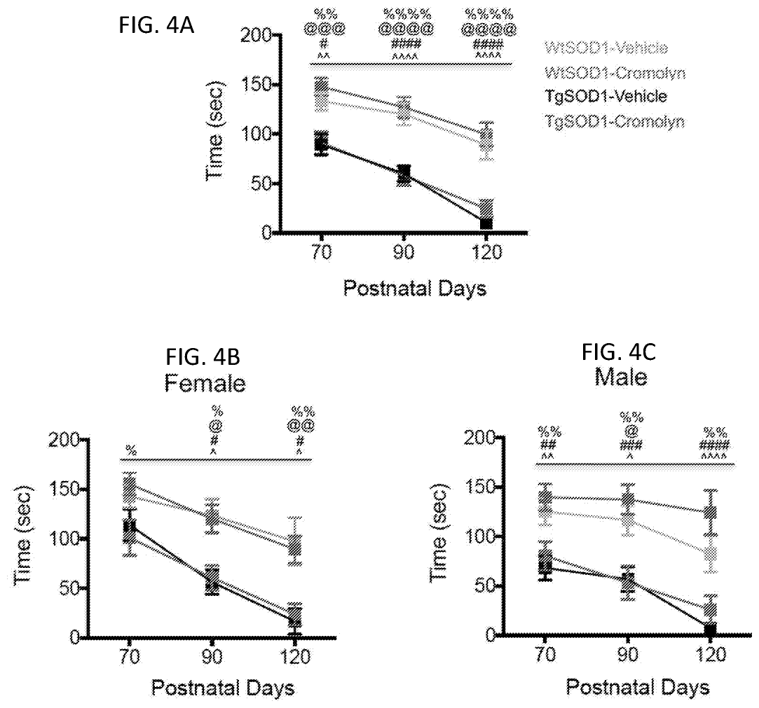

Motor coordination was assessed using the rotarod test. Two-way ANOVA demonstrated a significant effect of age [F(2, 361)=34.49, p<0.0001] and treatment [F(3, 361)=42.25, p<0.0001]. However, there was no significant age X treatment interaction on rotarod performance [F(6, 361)=0.704, p=0.646]. Tukey's post-hoc analysis revealed a significant difference between TgSOD1-Vehicle and both WtSOD1-Vehicle as well as WtSOD1-Cromolyn at P70, P90 and P120 (FIG. 4A). Similarly, post-hoc analysis revealed a significant decrease in rotarod performance between TgSOD1-Cromolyn group compared to WtSOD1-Vehicle and WtSOD1-Cromolyn at all time points (FIG. 4A). However, there was no difference in rotarod performance between the TgSOD1-Vehicle and TgSOD1-Cromolyn mice. In female mice, two-way ANOVA demonstrated a significant effect of age [F(2, 176)=19.04, p<0.0001] and treatment [F(3, 176)=14.48, p<0.0001]. However, there was no significant age X treatment interaction on rotarod performance [F(6, 176)=0.498, p=0.8086]. Tukey's post-hoc analysis revealed a significant difference between TgSOD1-Vehicle with both WtSOD1-Vehicle and WtSOD1-Cromolyn at P70, P90 and P120 (FIG. 4B). Similarly, post-hoc analysis revealed a significant decrease in rotarod performance between TgSOD1-Cromolyn group compared to WtSOD1-Vehicle and WtSOD1-Cromolyn at all time points (FIG. 4B). In male mice, two-way ANOVA demonstrated a significant effect of age [F(2, 169)=9.97, p<0.0001] and treatment [F(3, 169)=28.15, p<0.0001]. However, there was no significant age X treatment interaction on rotarod performance [F(6, 169)=0.561, p=0.7604]. Tukey's post-hoc analysis revealed a significant difference between TgSOD1-Vehicle with both WtSOD1-Vehicle and WtSOD1-Cromolyn at P70, P90 and P120 in male treated mice (FIG. 4C). Similarly, post-hoc analysis revealed a significant decrease in rotarod performance between male TgSOD1-Cromolyn group compared to WtSOD1-Vehicle and WtSOD1-Cromolyn at all time points (FIG. 4C). These data indicate that cromolyn treatment did not alter rotarod performance in TgSOD1 mice.

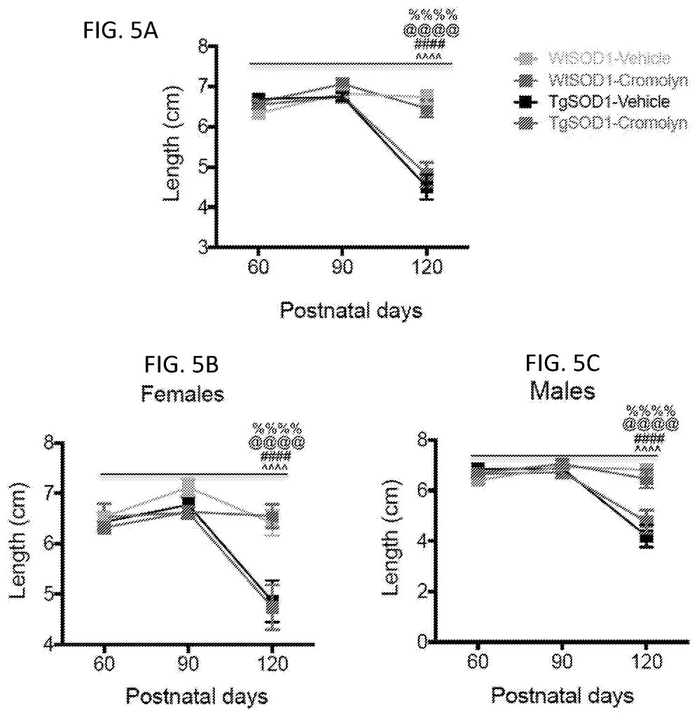

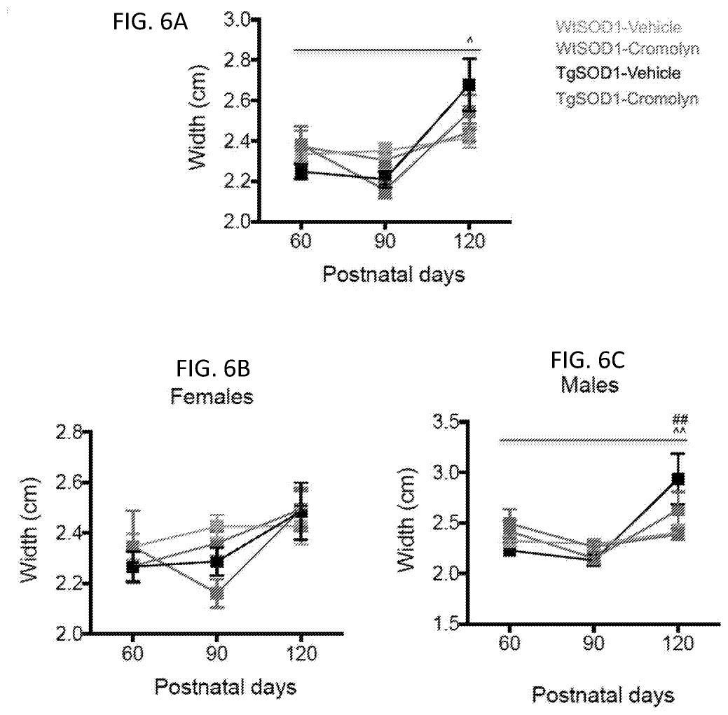

The effect of cromolyn treatment was also assessed on gait performance by measuring stride length and width. Two-way ANOVA demonstrated a significant effect of age [F(2, 403)=62.78, p<0.0001], treatment [F(3, 403)=18.96, p<0.0001], and age X treatment interaction on stride length [F(6, 403)=16.99, p<0.0001] in all groups. Tukey's post-hoc analysis revealed a significant decrease in stride length in TgSOD1-Vehicle compared with both wild-type groups at P120 (FIG. 5a). Similarly, post-hoc analysis revealed a significant decrease in stride length in TgSOD1-Cromolyn group compared to wild-type mice at P120 (FIG. 5A) suggesting that cromolyn treatment had no effect on stride length. In female mice, two-way ANOVA revealed a significant effect of age [F(2, 190)=27.85, p<0.0001], treatment [F(3, 190)=8.389, p<0.0001], and age X treatment interaction on stride length [F(6, 190)=6.278, p<0.0001]. Tukey's post-hoc analysis revealed a significant decrease in stride length in TgSOD1-Vehicle and TgSOD1-Cromolyn treated female mice compared with both wild-type groups at P120 (FIG. 5B). Similarly, in male mice, two-way ANOVA revealed a significant effect of age [F(2, 205)=37.9, p<0.0001], treatment [F(3, 205)=10.84, p<0.0001], and age X treatment interaction on stride length [F(6, 205)=10.86, p<0.0001]. Tukey's post-hoc analysis revealed a significant decrease in stride length in male TgSOD1-Vehicle and TgSOD1-Cromolyn treated mice compared with both wild-type groups at P120 (FIG. 5C). In addition, alterations in stride width were also assessed following treatment. In all groups, two-way ANOVA revealed that while there was no effect of treatment, there was a significant effect of age [F(2, 397)=18.3, p<0.0001] and age X treatment [F(6, 397)=3.159, p=0.0049] on stride width. Tukey's post-hoc analysis revealed a significant increase in stride width at P120 in TgSOD1-Vehicle group compared to WtSOD1-Vehicle (FIG. 6A). Two-way ANOVA analysis of female mice alone revealed a significant effect on age [F(2, 1935)=5.837, p=0.0035] only (FIG. 6B). Furthermore, two-way ANOVA in male mice revealed that while there was no effect of treatment, there was a significant effect of age [F(2, 189)=14.84, p<0.0001] and age X treatment [F(6, 189)=3.978, p=0.0009] on stride width. Post-hoc analysis revealed a significant increase in stride width in the TgSOD1-Vehicle treated mice compared to both wild-type groups (FIG. 6C). Thus, cromolyn treatment did not alter gait performance (e.g. stride length or width) in TgSOD1 mice.

### Effect of Cromolyn Sodium Treatment on Age at Paresis Onset and Survival

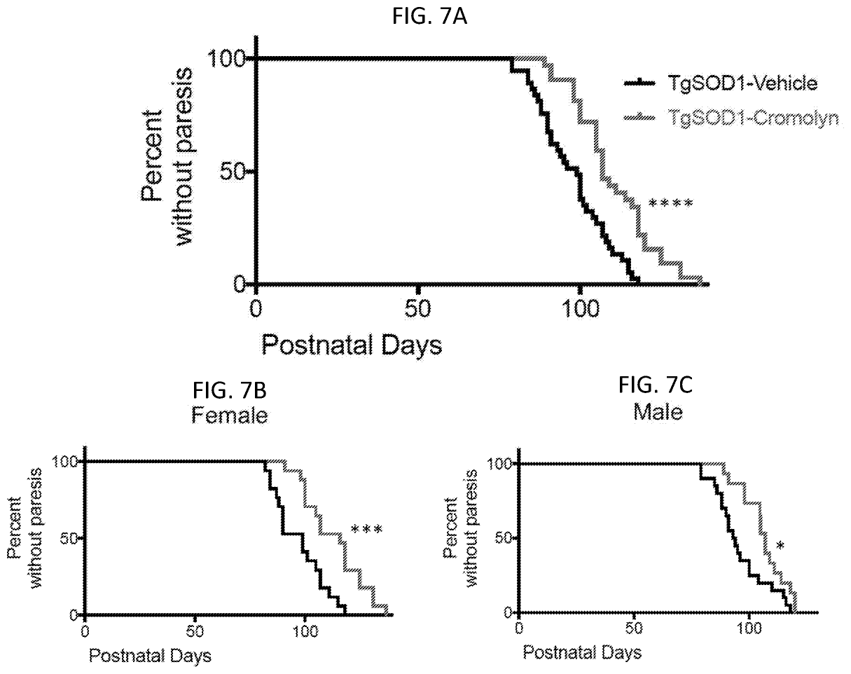

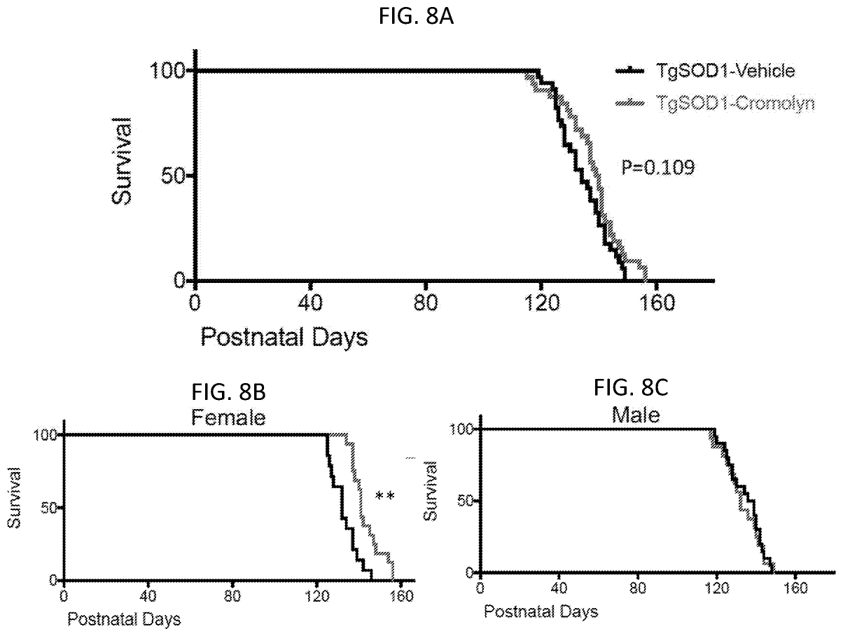

There was also a significant effect of cromolyn treatment on the onset of motor symptoms as measured by age at paresis onset (Mantel-Cox test, p<0.0001), with a median age of onset of 99 days for TgSOD1-Vehicle group and 107 days for the TgSOD1-Cromolyn group (FIG. 7A). Both female mice (Mantel-Cox test, p=0.0009) (FIG. 7B) and male mice (Mantel-Cox test, p=0.0193) (FIG. 7C) demonstrated a significant delay in the onset of motor symptoms following cromolyn treatment. While cromolyn treatment did not have a significant effect on survival in all treated mice (Mantel-Cox test, p=0.1096) or male mice alone (Mantel-Cox test, p<0.8831) (FIG. 8A, FIG. 8C), there was a significant effect of treatment on female survival (Mantel-Cox test, p=0.01) (FIG. 8B). These results suggest that cromolyn treatment delays the age at paresis in all TgSOD1 mice but only increases survival in female TgSOD1 mice.

### Cromolyn Treatment is Neuroprotective and Increases Survival of Lumbar Spinal Cord 224 Motor Neurons

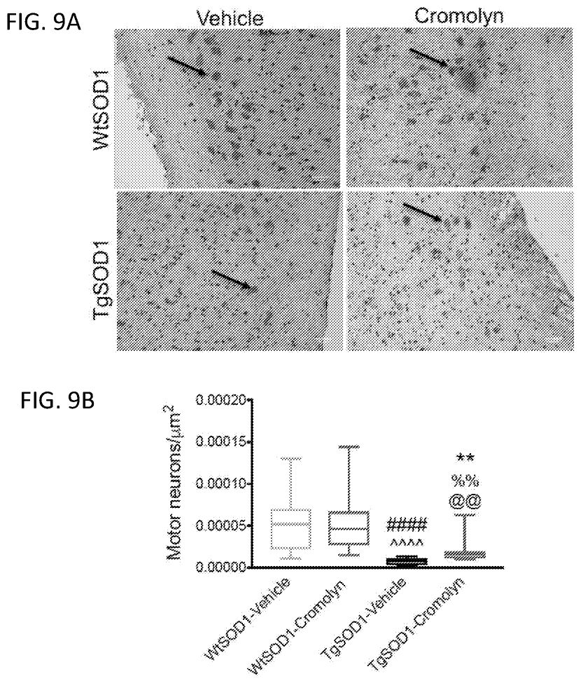

Next, we assessed the effect of cromolyn treatment on lumbar spinal cord motor neuron counts. Motor neurons of the lumbar spinal cord were visualized using hematoxylin and eosin (H&E) staining and a one-way ANOVA demonstrated a significant difference between the groups [F(4, 83)=60.31, p<0.0001]. Dunn's multiple comparisons test demonstrated a significant decrease in motor neuron counts in the TgSOD1-Vehicle group compared to WtSOD1-Vehicle (p<0.0001) and WtSOD1-Cromolyn (p<0.0001) groups. Furthermore, there was a significant decrease in motor neuron counts between TgSOD1-Cromolyn and WtSOD1-Vehicle (p=0.0077) and WtSOD1-Cromolyn (p=0.0081) groups. Importantly, motor neuron survival was significantly increased in the TgSOD1-Cromolyn compared to TgSOD1-Vehicle group (p=0.0033) (FIG. 9B), suggesting that cromolyn treatment is neuroprotective.

Cromolyn Treatment does not Alter Microgliosis in the Spinal Cord of TgSOD1 Mice

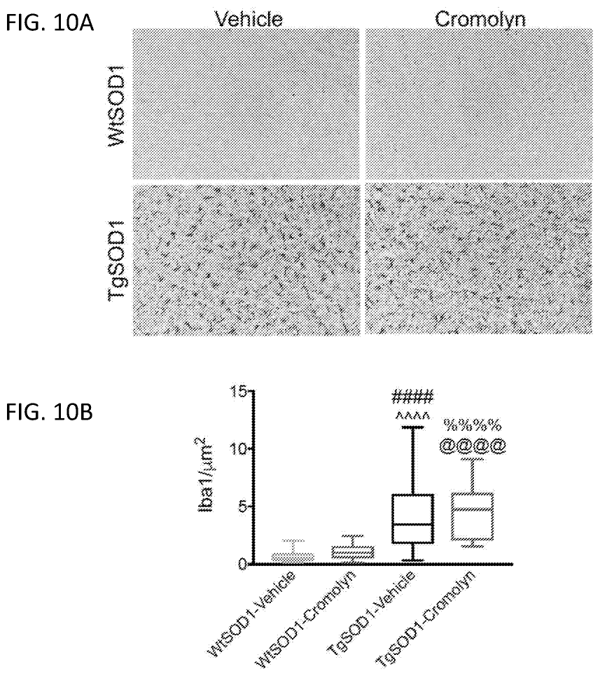

While acute treatment with cromolyn for one week was previously shown to lead to an increased number of microglia around β-amyloid plaques, chronic treatment significantly promoted microglial uptake and clearance of Aβ. Therefore, we assessed the effect of cromolyn treatment on microgliosis—by quantifying the percentage of microglia cells per lumbar spinal cord area. Microglial marker Iba1 was used to determine if similar effects could be observed after chronic treatment in the TgSOD1 mice. As reported previously, we found a significant increase in the percentage of Iba1-positive cell area in the lumbar spinal cord of vehicle treated TgSOD1 mice compared to WtSOD1 (FIG. 10A & FIG. 10B). In addition, there was a significant increase in the percentage of Iba1-positive cell area in the spinal cord of both vehicle and cromolyn-treated TgSOD1 compared to both wild-type groups as demonstrated by one-way ANOVA and Tukey's post-hoc analysis [F(4, 82)=53.12, p<0.0001] (FIG. 10B). However, there was no significant change in the percentage of Iba1-positive cell area in the lumbar spinal cord of TgSOD1-Cromolyn compared to TgSOD1-Vehicle (FIG. 10B). These data indicate that cromolyn treatment does not alter microgliosis in the lumbar spinal cord of TgSOD1 mice.

### Cromolyn Treatment Decreased the Levels of Pro-Inflammatory Cytokines/Chemokines in the Spinal Cord of TgSOD1 Mice

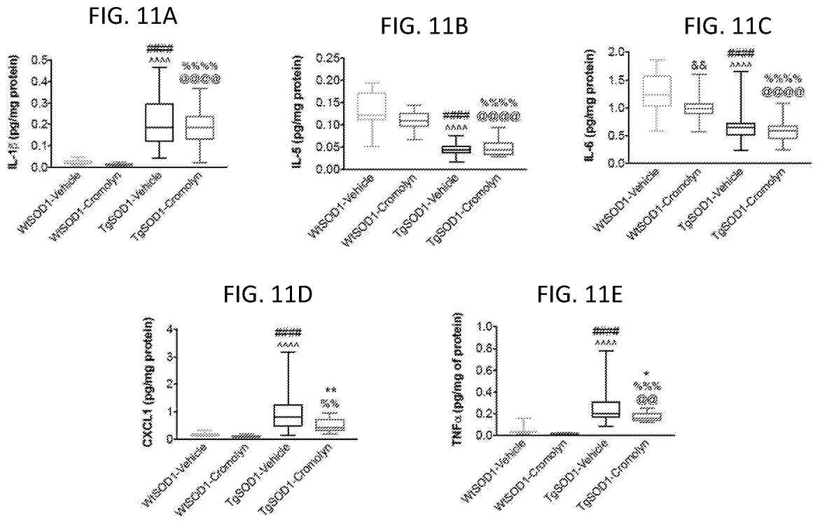

To assess the effects of cromolyn treatment on inflammation, we measured the levels of pro-inflammatory cytokines and chemokines in spinal cord lysates of mice by using the multi-spot assay system from Meso Scale Discovery. This assay allows for the simultaneous measurement of 10 cytokines and chemokines including: IFN-γ, IL-1β, IL-2, IL-4, IL-5, IL-6, CXCL1, IL-10, IL-12, and TNFα, which are known to be important in the neuroinflammatory response. Of these 10 cytokines and chemokines, we were able to successfully detect only 5 including IL-1b, IL-5, IL-6, CXCL1, and TNFα. One-way ANOVA and Tukey's post-hoc analysis revealed a significant difference in the levels of CXCL1 [F(3, 64)=18.15, p<0.0001], IL-1b [F(3, 130)=66.31, p<0.0001], IL-5 [F(3, 129)=129.9, p<0.0001], IL-6 [F(3, 135)=43.41, p<0.0001], and TNFα [F(3, 64)=27.94, p<0.0001], in the spinal cord of both TgSOD1-Vehicle and TgSOD1-Cromolyn groups compared to both wild-type groups (FIG. 11). There was a significant decrease in IL-6 (p<0.0001) and IL-5 (p<0.0001) levels between Tg and Wt groups (FIG. 11B & FIG. 11C). Importantly, there was a significant decrease in CXCL1 (p=0.0273) and TNFα (p=0.0273) levels in the TgSOD1-Cromolyn group compared to TgSOD1-Vehicle group (FIG. 11D & FIG. 11E), suggesting that cromolyn treatment decreased expression of pro-inflammatory cytokines and chemokines in the spinal cord of treated transgenic mice.

### Cromolyn Treatment Decreased the Levels of Pro-Inflammatory Cytokines/Chemokines in Plasma of TgSOD1 Mice

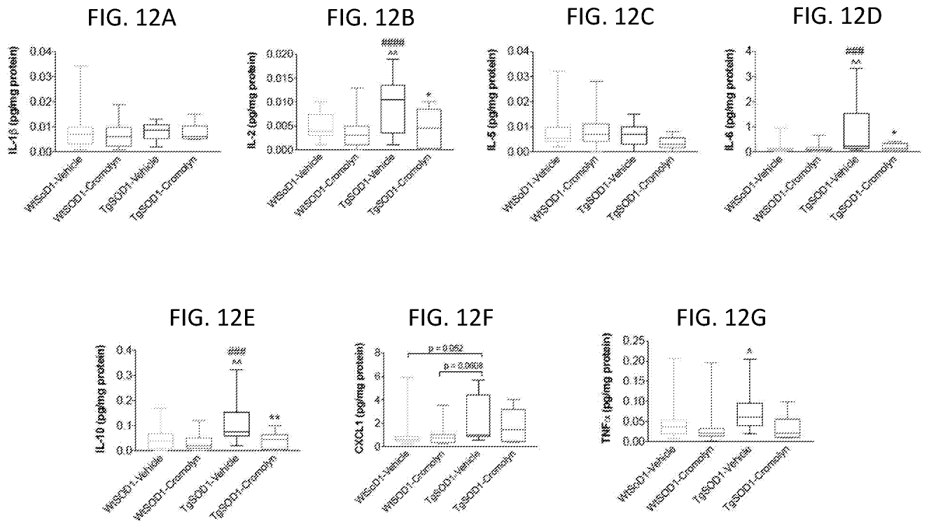

The same pro-inflammatory panel from Meso Scale Discovery was used to assess the levels of cytokines and chemokines in the plasma of a subset of mice (Females: 13 WtSOD1-Vehicle, 15 WtSOD1-Cromolyn, 6 TgSOD1-Vehicle, and 6 TgSOD1-Cromolyn; and Males: 14 WtSOD1-Vehicle, 10 WtSOD1-Cromolyn, 6 TgSOD1-Vehicle, 3 TgSOD1-Cromolyn). We were able to measure 7 of the 10 pro-inflammatory cytokines in plasma which included IL-1β, IL-2, IL-5, IL-6, CXCL1, IL-10, and TNFα. One-way ANOVA and Tukey's post-hoc analysis revealed a significant increase in IL-2 [F(3, 65)=7.731, p<0.0002], IL-6 [F(3, 63)=6.332, p<0.0008], and IL-10 [F(3, 65)=7.195, p<0.0003] levels in the plasma of TgSOD1-Vehicle compared to both WtSOD1-Vehicle and WtSOD1-Cromolyn groups (FIG. 12B, FIG. 12D & FIG. 12E). One-way ANOVA demonstrated a significant increase in CXCL1 levels [F(4, 69)=9.377, p<0.0247], and Tukey's post-hoc analysis revealed a significant increase in CXCL1 levels in TgSOD1-Vehicle mice compared to WtSOD1-Vehicle group (p=0.0318) and a trend towards an increase compared to WtSOD1-Cromolyn group (p=0.0847) (FIG. 12F). There was also a significant increase in TNFα levels [F(4, 67)=12.46, p<0.006], and post-hoc analysis revealed a significant increase in TNFα in TgSOD1-Vehicle group compared to WtSOD1-Cromolyn (p=0.0043) (FIG. 12G). There was no statistically significant difference in IL-1β and IL-5 levels between groups (FIG. 12A & FIG. 12C). Importantly, the levels of IL-2 (p=0.0211), IL-6 (p=0.0273), and IL-10 (p=0.0095) were significantly decreased in TgSOD1-Cromolyn group compared to TgSOD1-Vehicle group (FIG. 12B, FIG. 12D & FIG. 12E). Lastly, there was a trend towards a decrease in TNFα levels (p=0.110) in the TgSOD1-Cromolyn mice compared to the TgSOD1-Vehicle group (FIG. 12G). These results demonstrate that cromolyn treatment decreased the levels of cytokines in the plasma of TgSOD1 mice.

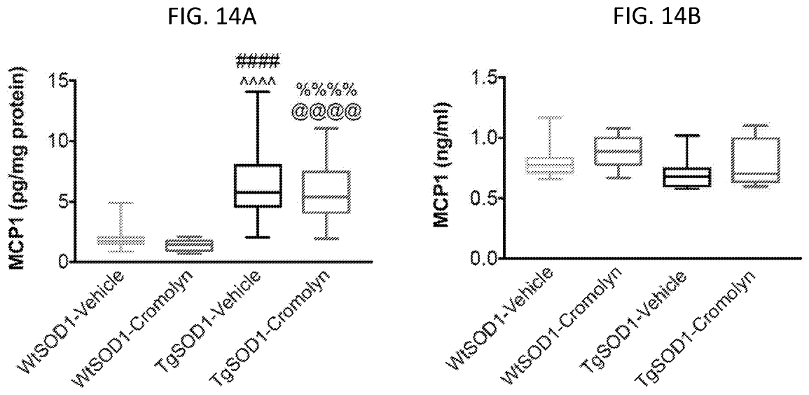

Previous studies have demonstrated that CCL2/MCP-1 levels are significantly increased in ALS. We were however unable to measure MCP-1 levels using the Meso Scale Discovery assay, as it was not represented on the specific panel that we used for our analysis. Therefore, we assessed the effect of cromolyn treatment on MCP-1 levels using an ELISA assay in the same spinal cord and plasma samples. One-way ANOVA and Tukey's post-hoc analysis revealed that the levels of MCP-1 were significantly increased in the spinal cord of TgSOD1-Cromolyn mice compared to both WtSOD1-Vehicle and WtSOD1-Cromolyn groups [F(3, 92)=46.24, p<0.0001] (FIG. 14A). However, there was no effect of cromolyn treatment on MCP-1 levels in the spinal cord of TgSOD1-Cromolyn compared to TgSOD1-Vehicle. Moreover, MCP-1 levels were not altered in the plasma in any of the groups [F(3, 32)=2.357, p<0.0902] (FIG. 14B). Thus, cromolyn treatment had no effect on MCP-1 levels in spinal cord or plasma of TgSOD1 mice.

### Cromolyn Treatment Increased GPR35 Levels in the Spinal Cord of TgSOD1 Mice

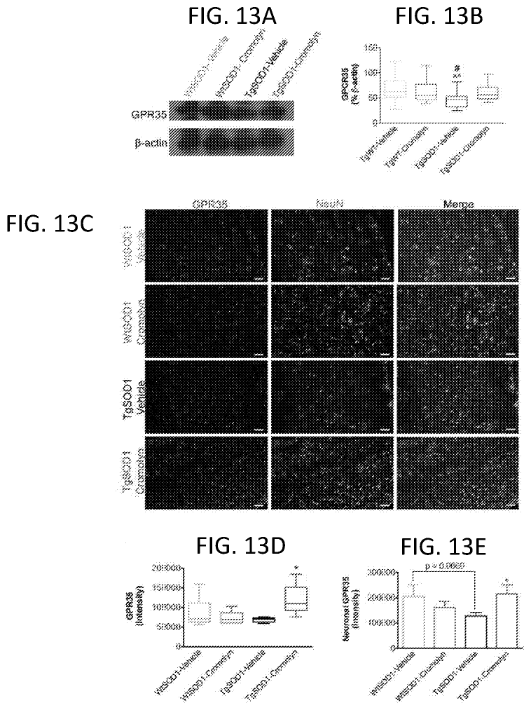

Cromolyn sodium is a potent agonist of the G-protein-coupled receptor 35 (GPR35), a receptor that has been suggested to play an important role in mast cell biology and a potential target for the treatment of asthma. Here, we assessed the effect of cromolyn on GPR35 expression using western blot analysis in the non-lumbar region of the spinal cord. One-way ANOVA demonstrated a significant difference in GRP35 levels [F(3, 70)=1.486, p<0.007] as measured by western blots and Tukey's post-hoc analysis revealed a significant decrease in GPR35 levels in TgSOD1-Vehicle group compared to WtSOD1-Vehicle (p=0.0047) and WtSOD1-Cromolyn group (p=0.0459) (FIG. 13A & FIG. 13B). Furthermore, there was no significant difference between the TgSOD1-Cromolyn group with either of the WtSOD1 groups. However, there was a trend towards an increase in GPR35 in the TgSOD1-Cromolyn group compared to the TgSOD1-Vehicle group (p=0.167) (FIG. 13B). Next, we confirmed these findings using immunofluorescence in the lumbar spinal cord. One-way ANOVA demonstrated a significant difference in GRP35 intensity [F(3, 19)=1.174, p<0.0348] in the lumbar spinal cord (FIG. 13C & FIG. 13D). Furthermore, Tukey's post-hoc analysis revealed a significant increase in GPR35 levels in TgSOD1-Cromolyn group compared to TgSOD1-Vehicle (p=0.0449) (FIG. 13D). GPR35 co-localized with the neuronal marker, NeuN, (FIG. 13C) and there was a significant increase in neuronal GPR35 levels in the TgSOD1-Cromolyn group compared to TgSOD1-Vehicle as measured by one-way ANOVA [F(3, 8)=0.375, p<0.0333] and post-hoc analysis (p=0.0411) (FIG. 13E). Together these findings suggest that cromolyn may provide its neuroprotective effects by regulating GPR35 expression and/or shifting GPR35 towards neuronal expression pattern in TgSOD1 mice.

What is claimed is:

1. A method of treating or slowing the progression of a disease or condition in a subject in need thereof comprising co-administering a first compound and a second compound,
wherein
the disease or condition is a neuron inflammation condition; and
the first compound and the second compound are independently
(a) a compound having formula (I): 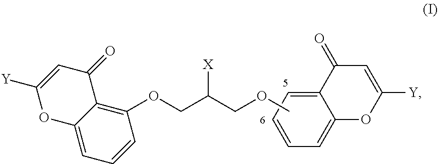
wherein
X is halide, hydroxyl, or OCO(C₁₋₈alkyl);
Y is CO₂R¹ or CH₂OR²;
R¹ is Li, Na, K, H, C₁₋₄alkyl, or —CH₂CO₂(C₁₋₅alkyl); and
R² is H or —C(O)(C₁₋₄alkyl),
or pharmaceutically acceptable salts thereof, or
(b) selected from bitolterol, fenoterol, isoprenaline, levosalbutamol, orciprenaline, pirbuterol, procaterol, ritodrine, salbutamol, terbutaline, arformoterol, bambuterol, clenbuterol, formoterol, salmeterol, abediterol, carmoterol, indacaterol, olodaterol, vilanterol, nedocromil, ketotifen, olopatadine, omalizumab, quercetine, mepolizumab, azelastine, and methylxanthines pemirolast, olopataidne, alfatoxin G₁, alfatoxin B₁, alfatoxin M₁, deoxynivalenol, zearalenone, ochratoxin A, fumonisin B₁, hydrolyzed fumonisin B₁, patulin, and ergotamine; or
(c) edaravone or riluzole; or
(d) selected from: 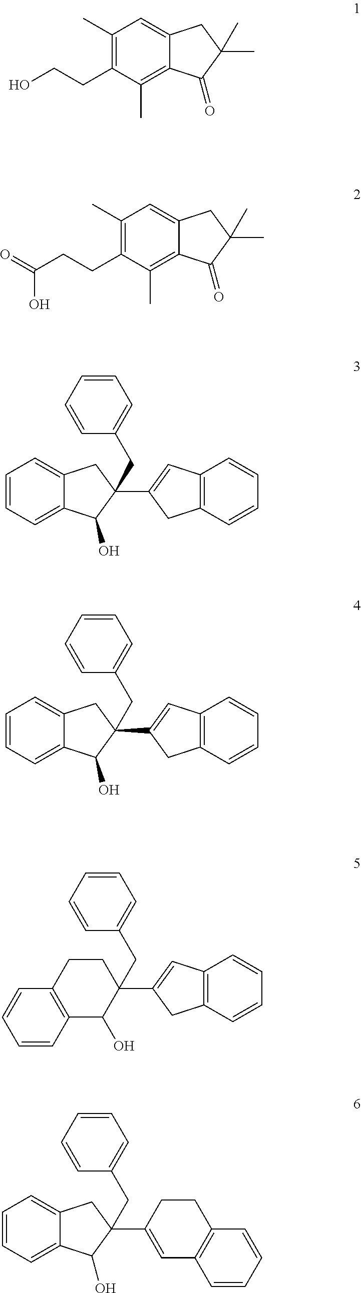 [SMILES: Cc1cc2c(c(C)c1CCO)C(=O)C(C)(C)C2 ; Cc1cc2c(c(C)c1CCC(=O)O)C(=O)C(C)(C)C2 ; O[C@H]1c2ccccc2C[C@]1(Cc1ccccc1)C1=Cc2ccccc2C1 ; O[C@H]1c2ccccc2C[C@@]1(Cc1ccccc1)C1=Cc2ccccc2C1 ; OC1c2ccccc2CCC1(Cc1ccccc1)C1=Cc2ccccc2C1 ; OC1c2ccccc2CC1(Cc1ccccc1)C1=Cc2ccccc2CC1] 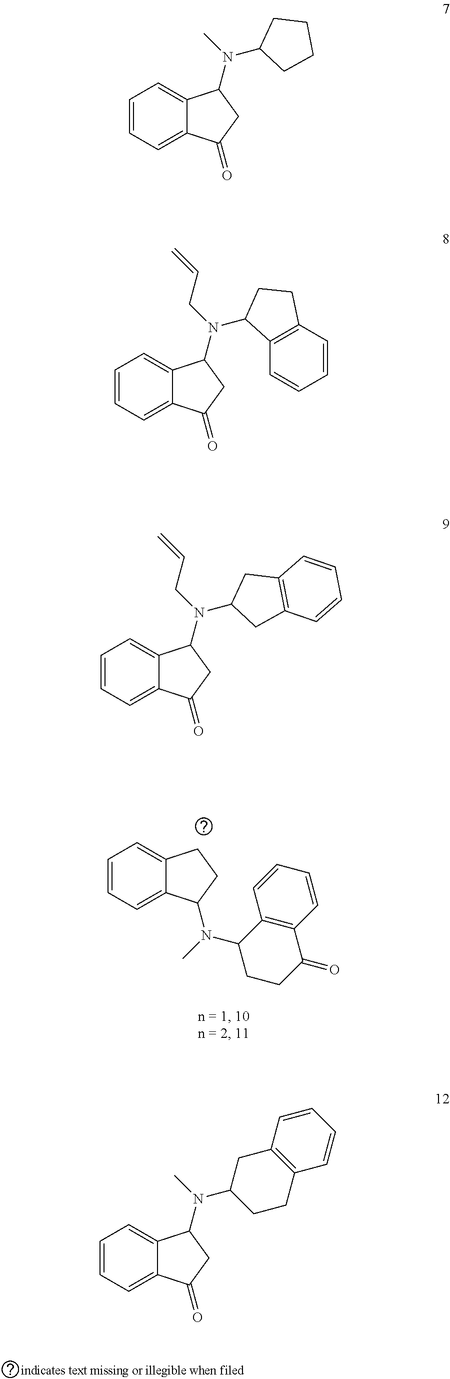 [SMILES: CN(C1CCCC1)C1CC(=O)c2ccccc21 ; C=CCN(C1CCc2ccccc21)C1CC(=O)c2ccccc21 ; C=CCN(C1Cc2ccccc2C1)C1CC(=O)c2ccccc21 ; CN(C1CCc2ccccc21)C1CCC(=O)c2ccccc21 ; CN(C1CCc2ccccc2C1)C1CC(=O)c2ccccc21]
or pharmaceutically acceptable salts thereof; or
(e) a non-steroidal anti-inflammatory drug (NSAID); or
(f) an anti-inflammatory peptide; and
the first compound and the second compound, taken together, are therapeutically effective.

2. The method according to claim 1, wherein the compound of formula (I) is selected from: 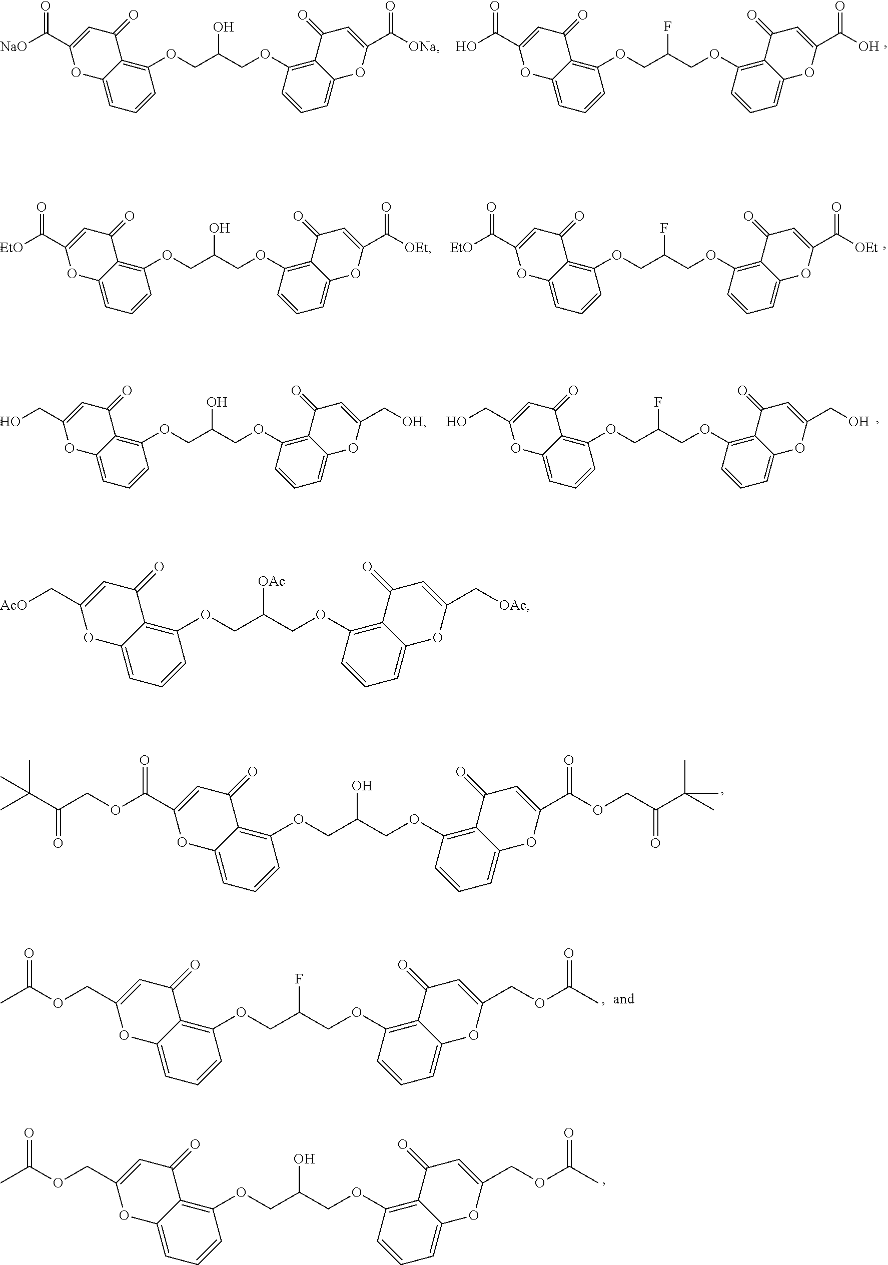
or pharmaceutically acceptable salts thereof.

3. The method of any one of claims 1-2, wherein the first compound is cromolyn sodium.

4. The method of any one of claims 1-3, wherein the second compound is a non-steroidal anti-inflammatory drug (NSAID).

5. The method of any one of claims 1-3, wherein the second compound is selected from acetylsalicylic acid, diflunisal, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, celecoxib, licofelone, hyperforin, and figwort.

6. The method of any one of claims 1-3, wherein the second compound is an anti-inflammatory small molecular peptide truncated from anti-inflammatory gene protein such as TREM2.

7. The method of any one of claims 1-6, wherein the neuron inflammation condition is ALS, autism spectrum disorder (ASD), ischemic stroke, and prion disease.

8. The method of any one of claims 1-6, wherein the neuron inflammation condition is ALS.

9. The method of any one of claims 1-6, wherein the neuron inflammation condition is a prion disease.

10. The method of any one of claims 1-9, wherein co-administration slows down or halts neuron damage for neurons located in the brain stem and/or the spinal cord, neurons, or motor neurons that affect voluntary body muscles.

11. The method of any one of claims 1-10, wherein the first compound or the second compound is administered subcutaneously, intravenously, intraperitoneally, orally or transdermally.

12. The method of any one of claims 1-10, wherein the first compound or the second compound is administered subcutaneously.

13. The method of any one of claims 1-10, wherein the first compound or the second compound is administered intravenously.

14. The method of any one of claims 1-10, wherein the first compound or the second compound is administered intraperitoneally.

15. The method of any one of claims 1-14, wherein the first compound and the second compound are administered in doses specifically tailored to lead to blood, brain, and CSF concentrations that allow the drugs to act as M1-to-M2 modifiers.
